(19) United States
(12) Patent Application Publication
(10) Pub. No.: US 2014/0303560 A1
(43) Pub. Date: Oct. 9, 2014

(54) Piston Driving Motor Arrangement

(71) Applicant: SANOFI-AVENTIS DEUTSCHLAND GMBH, Frankfurt am Main (DE)

(72) Inventors: Barry Yates, Warwickshire (GB); Aidan Michael O'Hare, Coventry (GB); Brian Molyneux, Warwickshire (GB)

(73) Assignee: SANOFI-AVENTIS DEUTSCHLAND GMBH, Frankfurt am Main (DE)

(21) Appl. No.: 14/358,691

(22) PCT Filed: Nov. 15, 2012
(86) PCT No.: PCT/EP2012/072795

§ 371 Date: May 15, 2014

(30) Foreign Application Priority Data
Nov. 18, 2011 (EP) 11189726.0

(51) Int. Cl. A61M 5/145; H02P 5/00; H02K 7/14; H02K 7/116

(52) U.S. Cl. CPC A61M 5/1452; H02K 7/116; H02P 5/00; H02K 7/14; A61M 2005/14506
USPC 604/154; 310/83; 318/5

(57) ABSTRACT

The invention is related to an apparatus comprising a first drive shaft, a second drive shaft, a motor component configured to receive a control signal, which control signal comprises a direction control signal, the motor component further configured to selectively drive the first drive shaft and the second drive shaft based on the received control signal, and further configured to drive the first drive shaft and the second drive shaft in a respective direction based on the direction control signal, such that a direction control signal for driving the first drive shaft in a first direction is a direction control signal for driving the second drive shaft in a direction opposite to the first direction. The invention is further related to a drug delivery device comprising an apparatus of the aforementioned kind.

### CROSS REFERENCE TO RELATED APPLICATIONS

The present application is a U.S. National Phase Application pursuant to 35 U.S.C. §371 of International Application No. PCT/EP2012/072795 filed Nov. 15, 2012, which claims priority to European Patent Application No. 11189726.0 filed Nov. 18, 2011. The entire disclosure contents of these applications are herewith incorporated by reference into the present application.

### FIELD OF INVENTION

The present patent application relates to a piston driving motor arrangement and more generally to an apparatus comprising a first drive shaft, a second drive shaft, and a motor component configured to receive a control signal, which control signal comprises a direction control signal, the motor component further configured to selectively drive the first drive shaft and the second drive shaft based on the received control signal.

### BACKGROUND

Such a piston driving motor arrangement can be used in a medical device for delivering at least two drug agents from separate reservoirs. Such drug agents may comprise a first and a second medicament. The medical device includes a dose setting mechanism for delivering the drug agents automatically or manually by the user. The medical device may use motor force to expel the drug agents from the reservoirs, for example from cartridges having a bung that is driven by a piston. The motor or motors driving the pistons may expel the drug agents sequentially or simultaneously.

The medical device can be an injector, for example a hand-held injector, especially a pen-type injector, that is an injector of the kind that provides for administration by injection of medicinal products from one or more multidose cartridges. In particular, the present invention relates to such injectors where a user may set the dose.

The drug agents may be contained in two or more multiple dose reservoirs, containers or packages, each containing independent (single drug compound) or pre-mixed (co-formulated multiple drug compounds) drug agents.

Certain disease states require treatment using one or more different medicaments. Some drug compounds need to be delivered in a specific relationship with each other in order to deliver the optimum therapeutic dose. The present patent application is of particular benefit where combination therapy is desirable, but not possible in a single formulation for reasons such as, but not limited to, stability, compromised therapeutic performance and toxicology.

For example, in some cases it may be beneficial to treat a diabetic with a long acting insulin (also may be referred to as the first or primary medicament) along with a glucagon-like peptide-1 such as GLP-1 or GLP-1 analog (also may be referred to as the second drug or secondary medicament).

Accordingly, there exists a need to provide devices for the delivery of two or more medicaments in a single injection or delivery step that is simple for the user to perform without complicated physical manipulations of the drug delivery device. The proposed drug delivery device provides separate storage containers or cartridge retainers for two or more active drug agents. These active drug agents are then combined and/or delivered to the patient during a single delivery procedure. These active agents may be administered together in a combined dose or alternatively, these active agents may be combined in a sequential manner, one after the other.

The drug delivery device also allows for the opportunity of varying the quantity of the medicaments. For example, one fluid quantity can be varied by changing the properties of the injection device (e.g., setting a user variable dose or changing the device's “fixed” dose). The second medicament quantity can be changed by manufacturing a variety of secondary drug containing packages with each variant containing a different volume and/or concentration of the second active agent.

The drug delivery device may have a single dispense interface. This interface may be configured for fluid communication with a primary reservoir and with a secondary reservoir of medicament containing at least one drug agent. The drug dispense interface can be a type of outlet that allows the two or more medicaments to exit the system and be delivered to the patient.

The combination of compounds from separate reservoirs can be delivered to the body via a double-ended needle assembly. This provides a combination drug injection system that, from a user's perspective, achieves drug delivery in a manner that closely matches the currently available injection devices that use standard needle assemblies. One possible delivery procedure may involve the following steps:

1. Attach a dispense interface to a distal end of the electro-mechanical injection device. The dispense interface comprises a first and a second proximal needle. The first and second needles pierce a first reservoir containing a primary compound and a second reservoir containing a secondary compound, respectively.

2. Attach a dose dispenser, such as a double-ended needle assembly, to a distal end of the dispense interface. In this manner, a proximal end of the needle assembly is in fluidic communication with both the primary compound and secondary compound.

3. Dial up/set a desired dose of the primary compound from the injection device, for example, via a graphical user interface (GUI).

4. After the user sets the dose of the primary compound, the micro-processor controlled control unit may determine or compute a dose of the secondary compound and preferably may determine or compute this second dose based on a previously stored therapeutic dose profile. It is this computed combination of medicaments that will then be injected by the user. The therapeutic dose profile may be user selectable. Alternatively, the user can dial or set a desired dose of the secondary compound.

5. Optionally, after the second dose has been set, the device may be placed in an armed condition. The optional armed condition may be achieved by pressing and/or holding an “OK” or an “Arm” button on a control panel. The armed condition may be provided for a predefined period of time during which the device can be used to dispense the combined dose.

6. Then, the user will insert or apply the distal end of the dose dispenser (e.g. a double ended needle assembly) into the desired injection site. The dose of the combination of the primary compound and the secondary compound (and potentially a third medicament) is administered by activating an injection user interface (e.g. an injection button).

Both medicaments may be delivered via one injection needle or dose dispenser and in one injection step. This offers a convenient benefit to the user in terms of reduced user steps compared to administering two separate injections.

The injection mechanism is implemented by a respective stopper being progressively moved in the respective cartridge, thereby forcing the liquid in the cartridge out. The stopper is mounted at the tip of a telescope piston rod, which extends when driven by a drive shaft. The drive shaft, in turn, is driven by a motor. There is a separate stopper, telescope piston rod, drive shaft and motor for each cartridge and therefore for each medicament to be injected.

In some embodiments, it may be required to inject the drugs or medicaments in a sequential order for a number of reasons. For example, the drugs shall not be mixed before injection for chemical or pharmacological reasons. Or the mechanic and/or electronic components of the medical device are not designed for simultaneous operation of two or more motors and piston rods. For example, the battery may not be able to provide sufficient current for two or more motors at the same time.

While the control software of the drug delivery device is configured to ensure a sequential and orderly injection of the medicaments strictly according to specification, in particular ruling out a simultaneous drug expulsion from more than one cartridge, there still remains the theoretical possibility that, because of a software bug or corrupt control signals, more than one telescope piston rod will advance simultaneously, thereby erroneously injecting the fluid from more than one cartridge simultaneously.

### SUMMARY

Thus it is an object of the invention to provide a reliable safeguard mechanism against the simultaneous injection of more than one medicament from the cartridges.

This object is solved by an apparatus comprising: a first drive shaft, a second drive shaft, a motor component configured to receive a control signal, which control signal comprises a direction control signal, the motor component further configured to selectively drive the first drive shaft and the second drive shaft based on the received control signal, and further configured to drive the first drive shaft and the second drive shaft in a respective direction based on the direction control signal, such that a direction control signal for driving the first drive shaft in a first direction is a direction control signal for driving the second drive shaft in a direction opposite to the first direction.

The control signal may be any electrical signal, including analog and digital, which is received by the motor component. In its most basic form, it may be a simple binary signal, transmitting either a “0” or a “1” signal, for example representing a motor “off” and “on” state. Also the direction control signal may be a binary signal, as it is part of the control signal.

The control signal may also be a signal transmitted in parallel or a serial signal, i.e. received on multiple signal lines or on a single signal line. In particular, the control signal may be transmitted on a signal bus.

The control signal may also carry information more complex than binary information, for example comprising any number of commands according to a protocol. The motor component is configured to receive the control signal and act on the direction control signal comprised by the control signal. The direction control signal indicates in which direction the first and the second drive shaft are driven. Other potential components of the control signal may determine further parameters of the motor components operation.

The direction control signal may be shared as only one drive shaft is driven at a time. By using a shared direction control for determining the direction of motion for both drive shafts, but associating it with opposed directions of motion for each drive shaft, a physical safeguard against simultaneous movement of both drive shafts in the same direction—in particular, simultaneous movement of both drive shafts in the direction to expel fluid from the cartridges—is implemented. Thereby such a simultaneous injection cannot occur, no matter how corrupt the control signals become either because of a software bug or because of electrical failures on the circuit board.

The first drive shaft is for expelling fluid from a first cartridge and the second drive shaft is for expelling fluid from a second cartridge. The drive shafts are driven by a motor component, which in turn is controlled by a control signal. This control signal may indicate velocity, torque, selective enablement and other control properties for the drive shaft and also comprises a direction control signal for determining the direction of rotation for each drive shaft. The motor component is configured such that it may, depending on the received control signal, apply different velocities, torques and other similar properties separately for each drive shaft—including the option of not driving one or both of the drive shafts at all. In any case, the same direction control signal is used for determining the direction of rotation for both drive shafts. This means that, according to the direction control signal, the direction of rotation of the first drive shaft is always opposite to the direction of rotation of the second drive shaft, notwithstanding the possibility of not having one or both drive shafts driven at any point in time.

A preferred embodiment is characterized in that the first drive shaft and the second drive shaft are arranged in parallel. The first drive shaft and the second drive shaft need not lie on the same axis, however they may however be oriented in the same direction.

A further preferred embodiment is characterized in that the apparatus further comprises a first mechanism for linearly moving a stopper, a second mechanism for linearly moving a stopper, a first gearing arrangement configured to couple the first drive shaft to the first mechanism for linearly moving a stopper, and a second gearing arrangement configured to couple the second drive shaft to the second mechanism for linearly moving a stopper. The first and second mechanisms for linearly moving a stopper and the first and second gearing arrangements are configured such that the first and second mechanism for linearly moving a stopper, respectively, engage in an extending action when the first or second drive shaft, respectively, is driven in a first direction and engages in a retracting action when the first or second drive shaft, respectively, is driven in a direction opposite to the first direction. A stopper is a component within a cartridge that creates a pressure on a fluid when a force acts on the stopper in a direction towards the opposite end of the cartridge. When for example a needle assembly is connected to the opposite end of the cartridge, the fluid is expelled through the needle assembly. The stopper moves within the cartridge as the fluid is expelled. The mechanism for linearly moving a stopper is a construct that translates a rotational movement, for example from a motor such as a direct current (DC) motor or a stepper motor, into a linear movement. The gearing arrangement transmits the rotational movement from its associated drive shaft to its associated mechanism for linearly moving a stopper.

In a further preferred embodiment, the first mechanism for linearly moving a stopper is configured to expel liquid from a first cartridge by engaging in the extending action and the second mechanism for linearly moving a stopper is configured to expel liquid from a second cartridge by engaging in the extending action.

In yet a further preferred embodiment, the first mechanism for linearly moving a stopper and the second mechanism for linearly moving a stopper are arranged in parallel. Thereby the direction of movement of each stopper is also parallel. However, the first mechanism for linearly moving a stopper and the second mechanism for linearly moving a stopper do not necessarily move their respective stopper on the same axis.

In a preferred embodiment, the first and second mechanisms for linearly moving a stopper comprise a first telescope piston rod and a second telescope piston rod, respectively.

In a further preferred embodiment, the motor component comprises a first motor configured to drive the first drive shaft, and a second motor configured to drive the second drive shaft. The first and second motors are those components that convert some other form of energy, for example electrical energy, into kinetic energy. The first and second motors can generate movement in one of at least two directions.

In yet a further preferred embodiment, the first and second motors are electric motors. This means that the first and second motors convert electrical energy into kinetic energy. The first motor may be a stepper motor. Alternatively or in addition, the second motor may be a stepper motor.

In another preferred embodiment, the first and second motors are each configured to selectively rotate in a clockwise direction or in a counter-clockwise direction. Each motor can rotate independently in one of the two directions. The direction of rotation in a clockwise direction or a counter-clockwise direction is defined based on the respective motors own alignment and orientation.

In a preferred embodiment, the first motor is arranged anti-parallel to the second motor such that a rotation of the first motor in the same direction as the rotation of the second motor causes the first drive shaft and the second drive shaft to be driven in opposite directions. An anti-parallel arrangement between the motors means that the first and the second motor each lies on a respective axis which is parallel, but need not be identical. Further, their orientation with respect to each other is reversed. That is, if the first motor is oriented in a first direction on its axis, the second motor is oriented in the opposite direction on its respective axis. By this anti-parallel arrangement of the first and second motors, an identical or equivalent gearing arrangement connecting each motor to one of two parallel drive shafts will ensure opposite directions of rotation for the drive shafts when both motors are rotated in the same direction, i.e. both are rotated either in a clockwise or a counter-clockwise direction from their own point of view.

In a further preferred embodiment, the apparatus further comprises a casing comprising a proximal end of the first drive shaft, a proximal end of the second drive shaft, the first motor, and the second motor, wherein the first drive shaft is shorter than the second drive shaft, wherein the first motor and the second motor are arranged at the proximal end of the first drive shaft, at an offset towards the axis of the second drive shaft and arranged in parallel to the second drive shaft. The casing need not be entirely closed, in particular a respective distal end of the first drive shaft and the second drive shaft may protrude from the casing. The first drive shaft and the second drive shaft may be arranged in parallel and with the same orientation. Because the first drive shaft is shorter than the second drive shaft, there exists available space which would have been taken up by a first drive shaft that was as long as the second drive shaft. The first and second motors may be arranged in this space. They may be arranged on a respective axis parallel to the second drive shaft. However, the axis of the two motors need not be identical to each other. The two motors are arranged preferably with an anti-parallel orientation to each other. By connecting each motor with a respective drive shaft through a respective gearing arrangement, a rotation of both motors either in a clockwise or counter-clockwise direction will cause the drive shafts to rotate in opposite directions.

In yet a further preferred embodiment, the apparatus comprises a control unit configured to generate the control signal and the direction control signal. This control unit may be a microcontroller or any other type of logic controller, for example a field programmable gate array, an application-specific integrated circuit, a complex programmable logic device, a programmable logic controller or any other device capable of supplying appropriate logic control signals.

In a still further preferred embodiment, the motor component comprises a first motor driver configured to drive the first motor based on the received control signal and the direction control signal, a second motor driver configured to drive the second motor based on the received control signal and the direction control signal, and at least one signal line configured to carry the control signal and the direction control signal from the control unit, wherein the first motor driver and the second motor driver are configured to at least partially share the at least one signal line such that they receive the identical direction control signal.

The first and second motor drivers are components controlling the operation of the first and second motors, respectively, with regard to activation, powering, direction of movement or rotation, torque, speed and any other adjustable parameter in the operation of the first and second motors. The control of the first and second motors is based on the received control signal. While all other parameters of the operation of the first and second motors may be provided separately for the first and second motor with the control signal, there is only a joint direction control signal for the first and second motors. The at least one signal line may be a parallel signal line or lines. The at least one signal line may alternatively or in addition be a serial signal line. The first and second motor driver share the at least one signal line at least partially and at least such that they physically receive the same direction control signal at the same time when receiving the control signal on the at least one signal line.

In a preferred embodiment, the first motor driver and the second motor driver are configured such that each receives the identical control signal and direction control signal from the shared at least one signal line.

In a further preferred embodiment, the control unit is configured to generate a first enable signal for selectively enabling the first motor driver and is configured to generate a second enable signal for selectively enabling the second motor driver. The first motor driver may be configured to only activate the first motor when it receives the first enable signal. The second motor driver may be configured to only activate the second motor when it receives the second enable signal. The first and second enable signals may be comprised in the control signal. The first and second enable signals may be transmitted via the at least one signal line. The first and second enable signals may also be transmitted to the first and second motor driver, respectively, by a dedicated signal line each. The first and second enable signals may also be transmitted to the first and second motor driver, respectively, by one signal line, the state of which determines which motor is enabled.

The invention is further directed at a drug delivery device comprising an apparatus according to any of the aforementioned embodiments.

### BRIEF DESCRIPTION OF THE DRAWINGS

These as well as other advantages of various aspects of the present invention will become apparent to those of ordinary skill in the art by reading the following detailed description, with appropriate reference to the accompanying drawings, in which:

FIG. 1 illustrates a perspective view of a delivery device with an end cap of the device removed;

FIG. 2 illustrates a perspective view of the delivery device distal end showing the cartridge;

FIG. 3 illustrates a perspective view of the delivery device illustrated in FIG. 1 or 2 with one cartridge retainer in an open position;

FIG. 4 illustrates a dispense interface and a dose dispenser that may be removably mounted on a distal end of the delivery device illustrated in FIG. 1;

FIG. 5 illustrates the dispense interface and the dose dispenser illustrated in FIG. 4 mounted on a distal end of the delivery device illustrated in FIG. 1;

FIG. 6 illustrates one arrangement of a needle assembly that may be mounted on a distal end of the delivery device;

FIG. 7 illustrates a perspective view of the dispense interface illustrated in FIG. 4;

FIG. 8 illustrates another perspective view of the dispense interface illustrated in FIG. 4;

FIG. 9 illustrates a cross-sectional view of the dispense interface illustrated in FIG. 4;

FIG. 10 illustrates an exploded view of the dispense interface illustrated in FIG. 4;

FIG. 11 illustrates a cross-sectional view of the dispense interface and needle assembly mounted onto a drug delivery device, such as the device illustrated in FIG. 1;

FIG. 12 illustrates a block diagram functional description of a control unit for operation of the drug delivery device illustrated in FIG. 4;

FIG. 13 illustrates a printed circuit board assembly of the drug delivery device illustrated in FIG. 4;

FIG. 14 illustrates a schematic view of a drive mechanism for use with the drug delivery device illustrated in FIG. 1;

FIG. 15 illustrates another schematic view of the drive mechanism illustrated in FIG. 14;

FIGS. 16a and 16b illustrate a motion detection system that may be used with the drive mechanism illustrated in FIG. 14;

FIG. 17 illustrates a schematic view of an alternative drive mechanism for use with the drug delivery device illustrated in FIG. 1;

FIG. 18 illustrates a schematic view of the alternative drive mechanism illustrated in FIG. 17 with certain elements removed;

FIG. 19 illustrates a schematic view of a telescope piston rod and gearing arrangement illustrated in FIG. 18;

FIG. 20 illustrates a schematic view of a telescope piston rod arrangement illustrated in FIG. 19;

FIG. 21 illustrates a schematic view of one piston rod arrangement illustrated in FIG. 19;

FIG. 22 illustrates a block diagram functional description of an exemplary arrangement of controlling the drive mechanism;

FIG. 23 illustrates a schematic view of an exemplary motor component as illustrated in FIG. 22.

### DETAILED DESCRIPTION

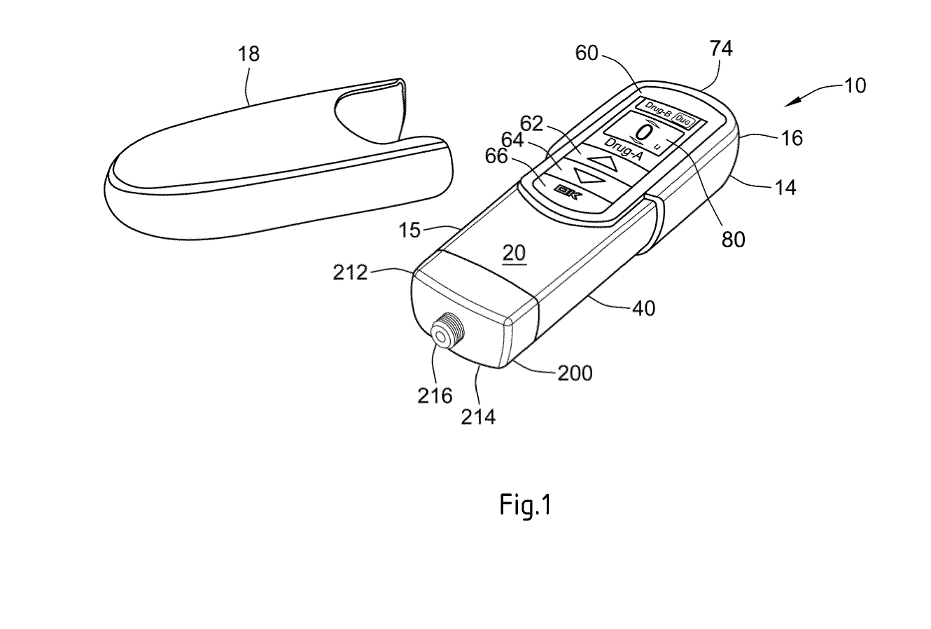

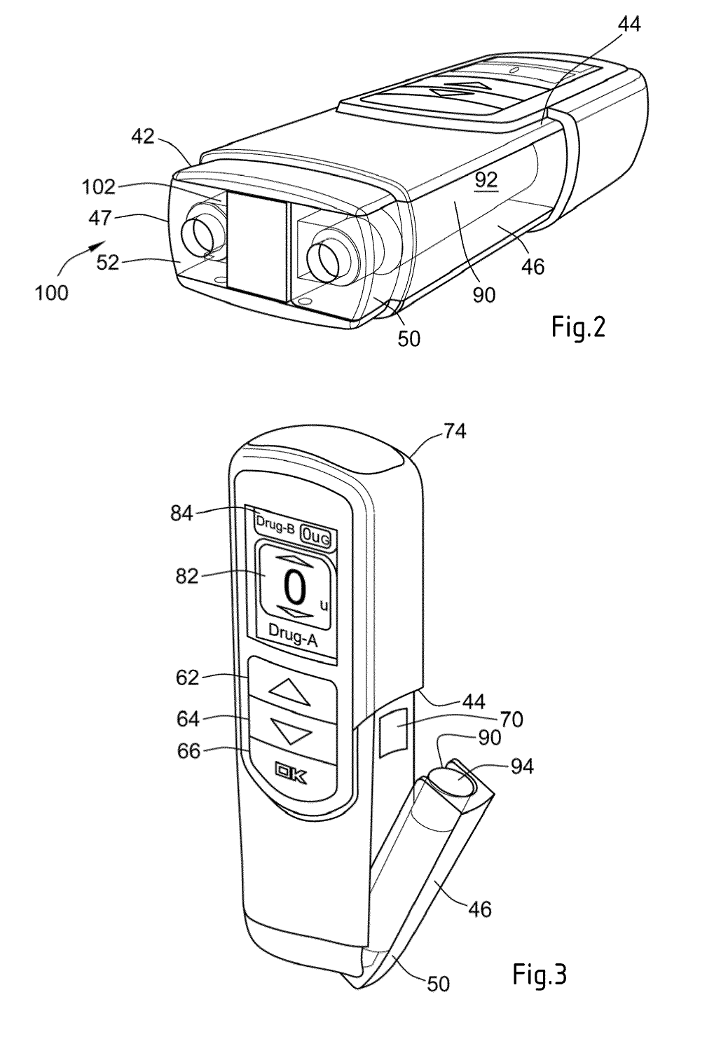

The drug delivery device illustrated in FIG. 1 comprises a main body 14 that extends from a proximal end 16 to a distal end 15. At the distal end 15, a removable end cap or cover 18 is provided. This end cap 18 and the distal end 15 of the main body 14 work together to provide a snap fit or form fit connection so that once the cover 18 is slid onto the distal end 15 of the main body 14, this frictional fit between the cap and the main body outer surface 20 prevents the cover from inadvertently falling off the main body.

The main body 14 contains a micro-processor control unit, an electro-mechanical drive train, and at least two medicament reservoirs. When the end cap or cover 18 is removed from the device 10 (as illustrated in FIG. 1), a dispense interface 200 is mounted to the distal end 15 of the main body 14, and a dose dispenser (e.g., a needle assembly) is attached to the interface. The drug delivery device 10 can be used to administer a computed dose of a second medicament (secondary drug compound) and a variable dose of a first medicament (primary drug compound) through a single needle assembly, such as a double ended needle assembly.

The drive train may exert a pressure on the bung of each cartridge, respectively, in order to expel the doses of the first and second medicaments. For example, a piston rod may push the bung of a cartridge forward a pre-determined amount for a single dose of medicament. When the cartridge is empty, the piston rod is retracted completely inside the main body 14, so that the empty cartridge can be removed and a new cartridge can be inserted.

A control panel region 60 is provided near the proximal end of the main body 14. Preferably, this control panel region 60 comprises a digital display 80 along with a plurality of human interface elements that can be manipulated by a user to set and inject a combined dose. In this arrangement, the control panel region comprises a first dose setting button 62, a second dose setting button 64 and a third button 66 designated with the symbol “OK.” In addition, along the most proximal end of the main body, an injection button 74 is also provided (not visible in the perspective view of FIG. 1).

The cartridge holder 40 can be removably attached to the main body 14 and may contain at least two cartridge retainers 50 and 52. Each retainer is configured so as to contain one medicament reservoir, such as a glass cartridge. Preferably, each cartridge contains a different medicament.

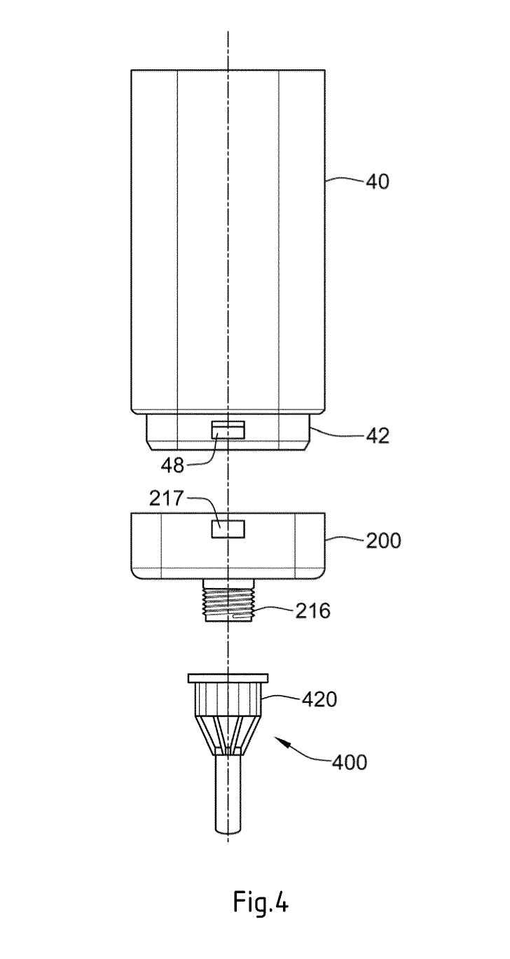

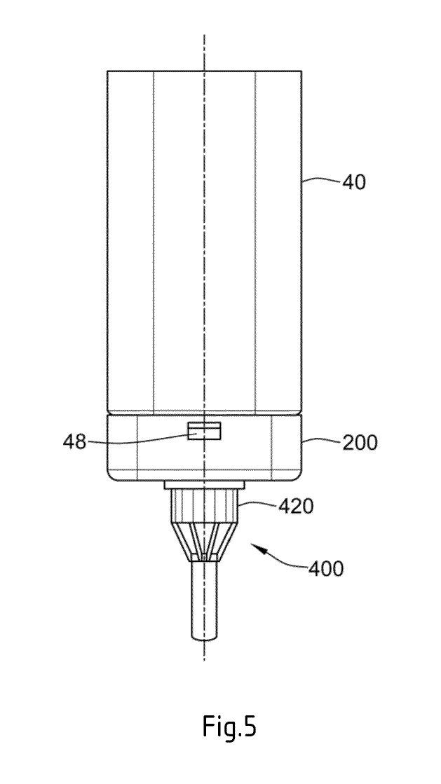

In addition, at the distal end of the cartridge holder 40, the drug delivery device illustrated in FIG. 1 includes a dispense interface 200. As will be described in relation to FIG. 4, in one arrangement, this dispense interface 200 includes a main outer body 212 that is removably attached to a distal end 42 of the cartridge housing 40. As can be seen in FIG. 1, a distal end 214 of the dispense interface 200 preferably comprises a needle hub 216. This needle hub 216 may be configured so as to allow a dose dispenser, such as a conventional pen type injection needle assembly, to be removably mounted to the drug delivery device 10.

Once the device is turned on, the digital display 80 shown in FIG. 1 illuminates and provides the user certain device information, preferably information relating to the medicaments contained within the cartridge holder 40. For example, the user is provided with certain information relating to both the primary medicament (Drug A) and the secondary medicament (Drug B).

As shown in FIG. 3, the first and second cartridge retainers 50, 52 may be hinged cartridge retainers. These hinged retainers allow user access to the cartridges. FIG. 3 illustrates a perspective view of the cartridge holder 40 illustrated in FIG. 1 with the first hinged cartridge retainer 50 in an open position. FIG. 3 illustrates how a user might access the first cartridge 90 by opening up the first retainer 50 and thereby having access to the first cartridge 90.

As mentioned above when discussing FIG. 1, a dispense interface 200 is coupled to the distal end of the cartridge holder 40. FIG. 4 illustrates a flat view of the dispense interface 200 unconnected to the distal end of the cartridge holder 40. A dose dispenser or needle assembly that may be used with the interface 200 is also illustrated and is provided in a protective outer cap 420.

In FIG. 5, the dispense interface 200 illustrated in FIG. 4 is shown coupled to the cartridge holder 40. The axial attachment means between the dispense interface 200 and the cartridge holder 40 can be any known axial attachment means to those skilled in the art, including snap locks, snap fits, snap rings, keyed slots, and combinations of such connections. The connection or attachment between the dispense interface and the cartridge holder may also contain additional features (not shown), such as connectors, stops, splines, ribs, grooves, pips, clips and the like design features, that ensure that specific hubs are attachable only to matching drug delivery devices. Such additional features would prevent the insertion of a non-appropriate secondary cartridge to a non-matching injection device.

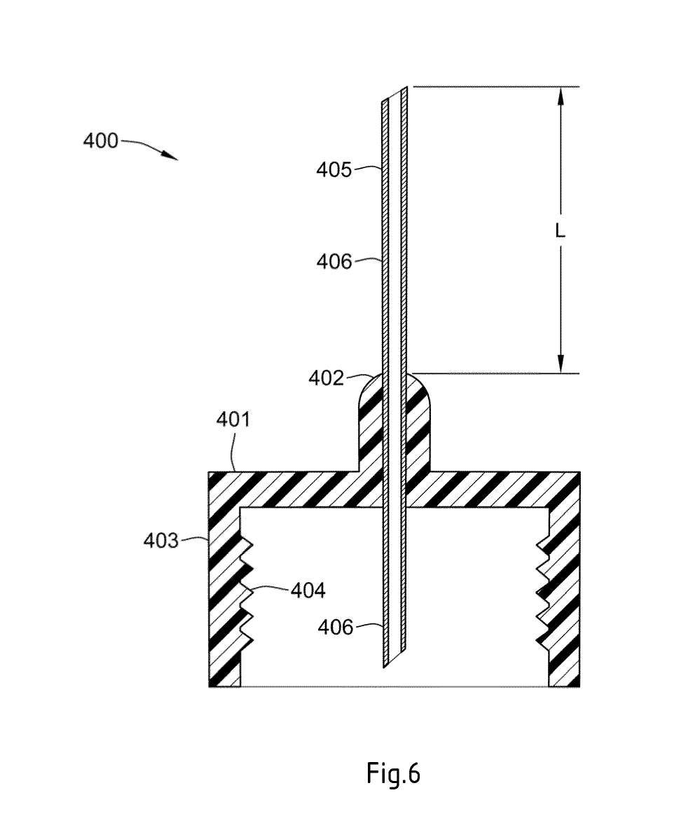

FIG. 5 also illustrates the needle assembly 400 and protective cover 420 coupled to the distal end of the dispense interface 200 that may be screwed onto the needle hub of the interface 200. FIG. 6 illustrates a cross sectional view of the double ended needle assembly 402 mounted on the dispense interface 200 in FIG. 5.

The needle assembly 400 illustrated in FIG. 6 comprises a double ended needle 406 and a hub 401. The double ended needle or cannula 406 is fixedly mounted in a needle hub 401. This needle hub 401 comprises a circular disk shaped element which has along its periphery a circumferential depending sleeve 403. Along an inner wall of this hub member 401, a thread 404 is provided. This thread 404 allows the needle hub 401 to be screwed onto the dispense interface 200 which, in one preferred arrangement, is provided with a corresponding outer thread along a distal hub. At a center portion of the hub element 401 there is provided a protrusion 402. This protrusion 402 projects from the hub in an opposite direction of the sleeve member. A double ended needle 406 is mounted centrally through the protrusion 402 and the needle hub 401. This double ended needle 406 is mounted such that a first or distal piercing end 405 of the double ended needle forms an injecting part for piercing an injection site (e.g., the skin of a user).

Similarly, a second or proximal piercing end 406 of the needle assembly 400 protrudes from an opposite side of the circular disc so that it is concentrically surrounded by the sleeve 403. In one needle assembly arrangement, the second or proximal piercing end 406 may be shorter than the sleeve 403 so that this sleeve to some extent protects the pointed end of the back sleeve. The needle cover cap 420 illustrated in FIGS. 4 and 5 provides a form fit around the outer surface 403 of the hub 401.

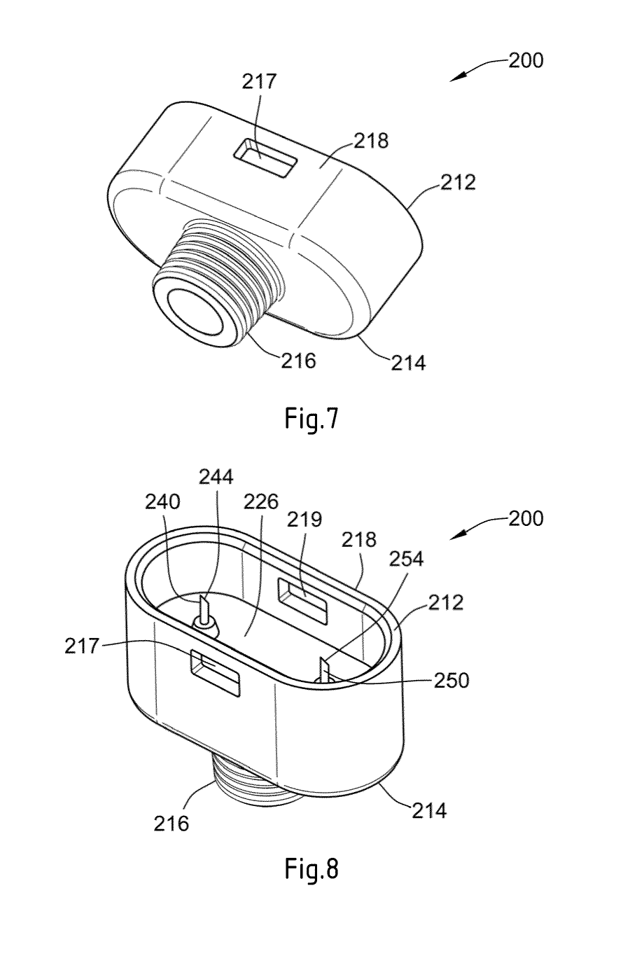

Referring now to FIGS. 4 to 11, one preferred arrangement of this interface 200 will now be discussed. In this one preferred arrangement, this interface 200 comprises:

a. a main outer body 210,

b. an first inner body 220,

c. a second inner body 230,

d. a first piercing needle 240,

e. a second piercing needle 250,

f. a valve seal 260, and

g. a septum 270.

The main outer body 210 comprises a main body proximal end 212 and a main body distal end 214. At the proximal end 212 of the outer body 210, a connecting member is configured so as to allow the dispense interface 200 to be attached to the distal end of the cartridge holder 40. Preferably, the connecting member is configured so as to allow the dispense interface 200 to be removably connected the cartridge holder 40. In one preferred interface arrangement, the proximal end of the interface 200 is configured with an upwardly extending wall 218 having at least one recess. For example, as may be seen from FIG. 8, the upwardly extending wall 218 comprises at least a first recess 217 and a second recess 219.

Preferably, the first and the second recesses 217, 219 are positioned within this main outer body wall so as to cooperate with an outwardly protruding member located near the distal end of the cartridge housing 40 of the drug delivery device 10. For example, this outwardly protruding member 48 of the cartridge housing may be seen in FIGS. 4 and 5. A second similar protruding member is provided on the opposite side of the cartridge housing. As such, when the interface 200 is axially slid over the distal end of the cartridge housing 40, the outwardly protruding members will cooperate with the first and second recess 217, 219 to form an interference fit, form fit, or snap lock. Alternatively, and as those of skill in the art will recognize, any other similar connection mechanism that allows for the dispense interface and the cartridge housing 40 to be axially coupled could be used as well.

The main outer body 210 and the distal end of the cartridge holder 40 act to form an axially engaging snap lock or snap fit arrangement that could be axially slid onto the distal end of the cartridge housing. In one alternative arrangement, the dispense interface 200 may be provided with a coding feature so as to prevent inadvertent dispense interface cross use. That is, the inner body of the hub could be geometrically configured so as to prevent an inadvertent cross use of one or more dispense interfaces.

A mounting hub is provided at a distal end of the main outer body 210 of the dispense interface 200. Such a mounting hub can be configured to be releasably connected to a needle assembly. As just one example, this connecting means 216 may comprise an outer thread that engages an inner thread provided along an inner wall surface of a needle hub of a needle assembly, such as the needle assembly 400 illustrated in FIG. 6. Alternative releasable connectors may also be provided such as a snap lock, a snap lock released through threads, a bayonet lock, a form fit, or other similar connection arrangements.

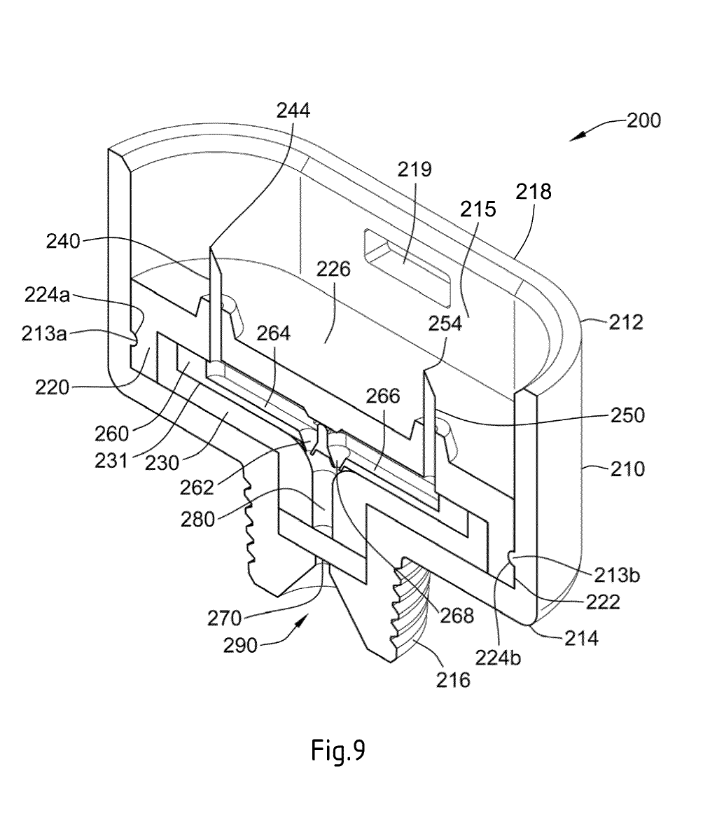

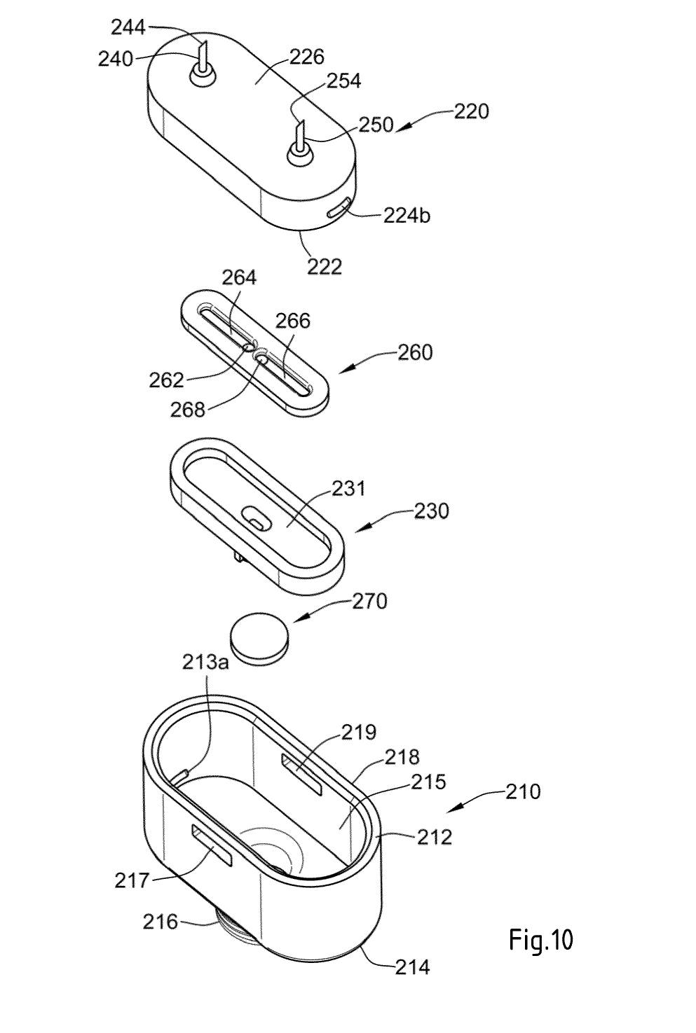

The dispense interface 200 further comprises a first inner body 220. Certain details of this inner body are illustrated in FIG. 8-11. Preferably, this first inner body 220 is coupled to an inner surface 215 of the extending wall 218 of the main outer body 210. More preferably, this first inner body 220 is coupled by way of a rib and groove form fit arrangement to an inner surface of the outer body 210. For example, as can be seen from FIG. 9, the extending wall 218 of the main outer body 210 is provided with a first rib 213a and a second rib 213b. This first rib 213a is also illustrated in FIG. 10. These ribs 213a and 213b are positioned along the inner surface 215 of the wall 218 of the outer body 210 and create a form fit or snap lock engagement with cooperating grooves 224a and 224b of the first inner body 220. In a preferred arrangement, these cooperating grooves 224a and 224b are provided along an outer surface 222 of the first inner body 220.

In addition, as can be seen in FIG. 8-10, a proximal surface 226 near the proximal end of the first inner body 220 may be configured with at least a first proximally positioned piercing needle 240 comprising a proximal piercing end portion 244. Similarly, the first inner body 220 is configured with a second proximally positioned piercing needle 250 comprising a proximally piercing end portion 254. Both the first and second needles 240, 250 are rigidly mounted on the proximal surface 226 of the first inner body 220.

Preferably, this dispense interface 200 further comprises a valve arrangement. Such a valve arrangement could be constructed so as to prevent cross contamination of the first and second medicaments contained in the first and second reservoirs, respectively. A preferred valve arrangement may also be configured so as to prevent back flow and cross contamination of the first and second medicaments.

In one preferred system, dispense interface 200 includes a valve arrangement in the form of a valve seal 260. Such a valve seal 260 may be provided within a cavity 231 defined by the second inner body 230, so as to form a holding chamber 280. Preferably, cavity 231 resides along an upper surface of the second inner body 230. This valve seal comprises an upper surface that defines both a first fluid groove 264 and second fluid groove 266. For example, FIG. 9 illustrates the position of the valve seal 260, seated between the first inner body 220 and the second inner body 230. During an injection step, this seal valve 260 helps to prevent the primary medicament in the first pathway from migrating to the secondary medicament in the second pathway, while also preventing the secondary medicament in the second pathway from migrating to the primary medicament in the first pathway. Preferably, this seal valve 260 comprises a first non-return valve 262 and a second non-return valve 268. As such, the first non-return valve 262 prevents fluid transferring along the first fluid pathway 264, for example a groove in the seal valve 260, from returning back into this pathway 264. Similarly, the second non-return valve 268 prevents fluid transferring along the second fluid pathway 266 from returning back into this pathway 266.

Together, the first and second grooves 264, 266 converge towards the non-return valves 262 and 268 respectively, to then provide for an output fluid path or a holding chamber 280. This holding chamber 280 is defined by an inner chamber defined by a distal end of the second inner body both the first and the second non return valves 262, 268 along with a pierceable septum 270. As illustrated, this pierceable septum 270 is positioned between a distal end portion of the second inner body 230 and an inner surface defined by the needle hub of the main outer body 210.

The holding chamber 280 terminates at an outlet port of the interface 200. This outlet port 290 is preferably centrally located in the needle hub of the interface 200 and assists in maintaining the pierceable seal 270 in a stationary position. As such, when a double ended needle assembly is attached to the needle hub of the interface (such as the double ended needle illustrated in FIG. 6), the output fluid path allows both medicaments to be in fluid communication with the attached needle assembly.

The hub interface 200 further comprises a second inner body 230. As can be seen from FIG. 9, this second inner body 230 has an upper surface that defines a recess, and the valve seal 260 is positioned within this recess. Therefore, when the interface 200 is assembled as shown in FIG. 9, the second inner body 230 will be positioned between a distal end of the outer body 210 and the first inner body 220. Together, second inner body 230 and the main outer body hold the septum 270 in place. The distal end of the inner body 230 may also form a cavity or holding chamber that can be configured to be fluid communication with both the first groove 264 and the second groove 266 of the valve seal.

Axially sliding the main outer body 210 over the distal end of the drug delivery device attaches the dispense interface 200 to the multi-use device. In this manner, a fluid communication may be created between the first needle 240 and the second needle 250 with the primary medicament of the first cartridge and the secondary medicament of the second cartridge, respectively.

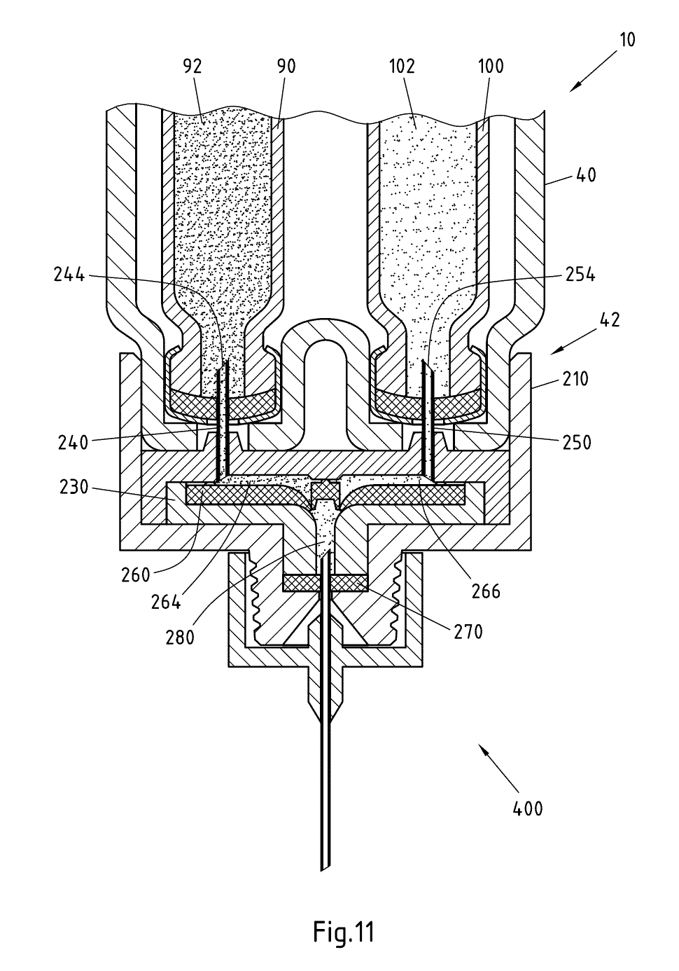

FIG. 11 illustrates the dispense interface 200 after it has been mounted onto the distal end 42 of the cartridge holder 40 of the drug delivery device 10 illustrated in FIG. 1. A double ended needle 400 is also mounted to the distal end of this interface. The cartridge holder 40 is illustrated as having a first cartridge containing a first medicament and a second cartridge containing a second medicament.

When the interface 200 is first mounted over the distal end of the cartridge holder 40, the proximal piercing end 244 of the first piercing needle 240 pierces the septum of the first cartridge 90 and thereby resides in fluid communication with the primary medicament 92 of the first cartridge 90. A distal end of the first piercing needle 240 will also be in fluid communication with a first fluid path groove 264 defined by the valve seal 260.

Similarly, the proximal piercing end 254 of the second piercing needle 250 pierces the septum of the second cartridge 100 and thereby resides in fluid communication with the secondary medicament 102 of the second cartridge 100. A distal end of this second piercing needle 250 will also be in fluid communication with a second fluid path groove 266 defined by the valve seal 260.

FIG. 11 illustrates a preferred arrangement of such a dispense interface 200 that is coupled to a distal end 15 of the main body 14 of drug delivery device 10. Preferably, such a dispense interface 200 is removably coupled to the cartridge holder 40 of the drug delivery device 10.

As illustrated in FIG. 11, the dispense interface 200 is coupled to the distal end of a cartridge housing 40. This cartridge holder 40 is illustrated as containing the first cartridge 90 containing the primary medicament 92 and the second cartridge 100 containing the secondary medicament 102. Once coupled to the cartridge housing 40, the dispense interface 200 essentially provides a mechanism for providing a fluid communication path from the first and second cartridges 90, 100 to the common holding chamber 280. This holding chamber 280 is illustrated as being in fluid communication with a dose dispenser. Here, as illustrated, this dose dispenser comprises the double ended needle assembly 400. As illustrated, the proximal end of the double ended needle assembly is in fluid communication with the chamber 280.

In one preferred arrangement, the dispense interface is configured so that it attaches to the main body in only one orientation, that is it is fitted only one way round. As such as illustrated in FIG. 11, once the dispense interface 200 is attached to the cartridge holder 40, the primary needle 240 can only be used for fluid communication with the primary medicament 92 of the first cartridge 90 and the interface 200 would be prevented from being reattached to the holder 40 so that the primary needle 240 could now be used for fluid communication with the secondary medicament 102 of the second cartridge 100. Such a one way around connecting mechanism may help to reduce potential cross contamination between the two medicaments 92 and 102.

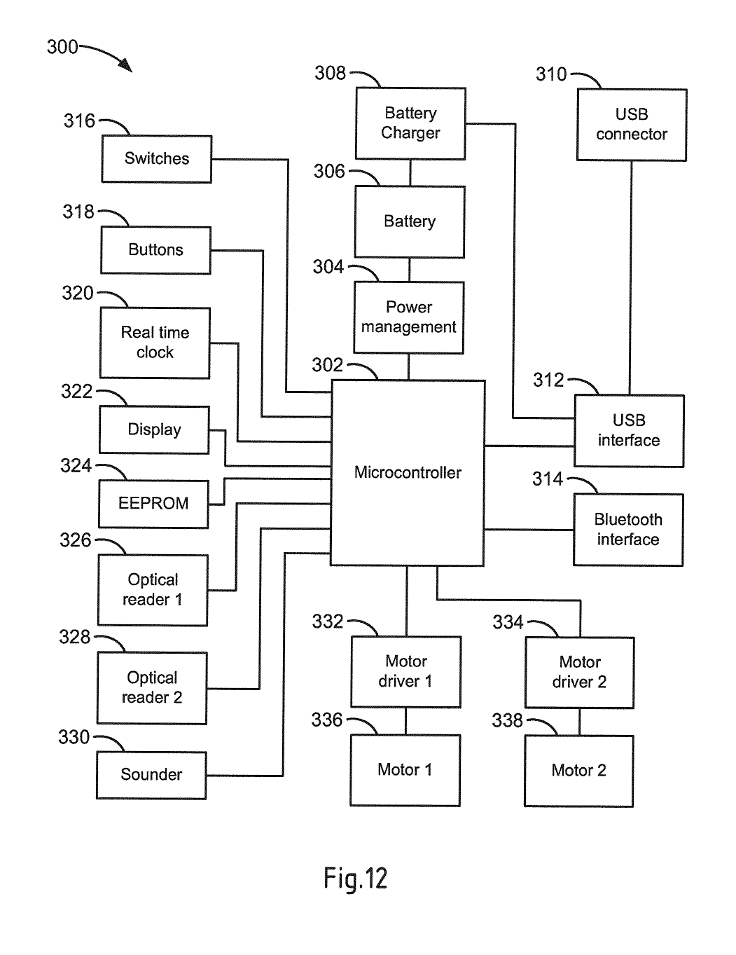

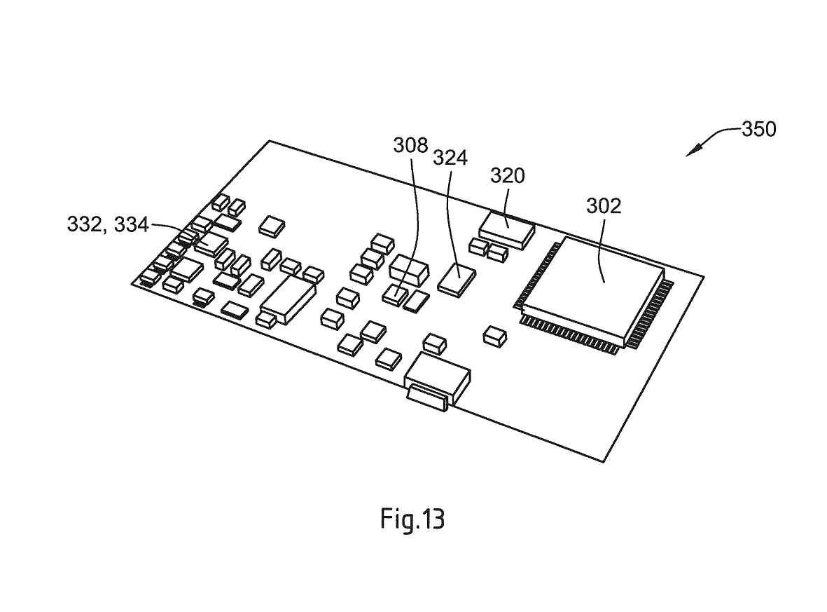

FIG. 12 illustrates a functional block diagram of a control unit to operate and control the drug delivery device illustrated in FIG. 1. FIG. 13 illustrates one arrangement of a printed circuit board (PCB) or printed circuit board assembly (PCBA) 350 that may comprise certain portions of the control unit illustrated in FIG. 12.

Referring now to both FIGS. 12 and 13, it may be seen that the control unit 300 comprises a microcontroller 302. Such a microcontroller may comprise a Freescale MCF51JM microcontroller. The microcontroller 302 is configured to control the electronic system for the drug delivery device 10 and comprises a microprocessor with an arithmetic logic unit (ALU). It may include at least one or more of the following peripheral circuits:
internal analogue to digital converters,
general purpose digital I/O lines,
one or more output digital Pulse Width Modulated (PWM) signals,
an internal USB module.

In one arrangement, a USB protection circuit such as ON-Semi NUP3115 may be implemented. In such an implementation, the actual USB communications may be provided on board the microcontroller 302.

The control unit further comprises a power management module 304 coupled to the microcontroller 302 and other circuit elements. The power management module 304 receives a supply voltage from a main power source such as the battery 306 and regulates this supply voltage to a plurality of voltages required by other circuit components of the control unit 300. In one preferred control unit arrangement, switched mode regulation (by means of a National Semiconductor LM2735) is used to step up the battery voltage to 6V, with linear regulation to generate other supply voltages required by the control unit 300.

The battery 306 provides power to the control unit 300 and is preferably supplied by a single lithium-ion or lithium-polymer cell. This cell may be encapsulated in a battery pack that contains safety circuitry to protect against overheating, overcharging and excessive discharge. The battery pack may also optionally contain coulomb counting technology to obtain an improved estimate of remaining battery charge.

A battery charger 308 may be coupled to the battery 306. One such battery charger may be based on Freescale Semiconductor MC34675 along with other supporting software and hardware modules. In an alternative embodiment, a Texas Instruments (TI) BQ24150 may also be used. In one preferred arrangement, the battery charger 308 takes energy from the external wired connection to the drug delivery device 10 and uses it to charge the battery 306. The battery charger 308 can also be used to monitor the battery voltage and charge current to control battery charging. The battery charger 308 can also be configured to have bidirectional communications with the microcontroller 302 over a serial bus. The charge status of the battery 306 may be communicated to the microcontroller 302 as well. The charge current of the battery charger may also be set by the microcontroller 302.

The control unit may also comprise a USB connector 310. A custom design of a connector or a micro USB-AB connector may be used for wired communications and to supply power to the device.

The control unit may also comprise a USB interface 312. This interface 312 may be external to the microcontroller 302. The USB interface 312 may have USB master and/or USB device capability. The USB interface 312 may also provide USB on-the-go functionality. The USB interface 312 external to the microcontroller also provides transient voltage suppression on the data lines and VBUS line.

An external Bluetooth interface 314 may also be provided. The Bluetooth interface 314 is preferably external to the microcontroller 302 and communicates with this controller 302 using a data interface.

Preferably, the control unit further comprises a plurality of switches 316. In the illustrated arrangement, the control unit 300 may comprise eight switches 316 and these switches may be distributed around the device. These switches 316 may be used to detect and or confirm at least the following:

a. Whether the dispense interface 200 has been properly attached to the drug delivery device 10;

b. Whether the removable cap 18 has been properly attached to the main body 20 of the drug delivery device 10;

c. Whether the first cartridge retainer 50 of the cartridge holder 40 for the first cartridge 90 has been properly closed;

d. Whether the second cartridge retainer 52 of the cartridge holder 40 for the second cartridge 100 has been properly closed;

e. To detect the presence of the first cartridge 90;

f. To detect the presence of the second cartridge 100;

g. To determine the position of the stopper 94 in the first cartridge 90; and

h. To determine the position of the stopper 104 in the second cartridge 100.

These switches 316 are connected to digital inputs, for example to general purpose digital inputs, on the microcontroller 302. Preferably, these digital inputs may be multiplexed in order to reduce the number of input lines required. Interrupt lines may also be used appropriately on the microcontroller 302 so as to ensure timely response to changes in switch status.

In addition, and as described in greater detail above, the control unit may also be operatively coupled to a plurality of human interface elements or push buttons 318. In one preferred arrangement, the control unit 300 comprises eight push buttons 318 and these are used on the device for user input for the following functions:

a. Dose dial up;

b. Dose dial down;

c. Sound level;

d. Dose;

e. Eject;

f. Prime;

g. Back; and

h. OK.

These buttons 318 are connected to digital inputs, for example to general purpose digital inputs, on the microcontroller. Again, these digital inputs may be multiplexed so as to reduce the number of input lines required. Interrupt lines will be used appropriately on the microcontroller to ensure timely response to changes in switch status. In an example embodiment, the function of one or more buttons may be replaced by a touch screen.

The microcontroller 302 may comprise a real time clock 320. In an alternative embodiment, the control unit 300 comprises the real time clock 320. In another alternative embodiment, the real time clock is in a separate chip or circuit, for example an Epson RX4045 SA. The real-time clock 320 may communicate with the microcontroller 302 using a serial bus such as a serial peripheral interface (SPI) or similar.

A digital display module 322 in the device preferably uses LCD or OLED technology and provides a visual signal to the user. The display module incorporates the display itself and a display driver integrated circuit. This circuit communicates with the microcontroller 302 using a serial peripheral interface or parallel bus.

The control unit 300 also comprises a memory device, for example volatile and non-volatile memory. Volatile memory may be random access memory (RAM), for example static RAM or dynamic RAM and/or the like, as working memory of microcontroller 302. Non-volatile memory may be read only memory (ROM), FLASH memory or electrically erasable programmable read-only memory (EEPROM), such as an EEPROM 324. Such an EEPROM may comprise an ON Semiconductor CAT25128. In an alternative embodiment, the EEPROM may comprise an Atmel AT25640. The EEPROM may be used to store system parameters and history data. This memory device 324 communicates with the processor 302 using a serial peripheral interface bus.

In an alternative embodiment, the control unit 300 further comprises a first and a second optical reader 326, 328. Such optical readers may comprise Avago ADNS3550. These optical readers 326, 328 may be optional for the drug delivery device 10 and are, as described above, used to read information from a cartridge when such a cartridge is inserted into either the first or the second cartridge retainers 50, 52. Preferably, a first optical reader is dedicated for the first cartridge and the second optical reader is dedicated for the second cartridge. An integrated circuit designed for use in optical computer mice may be used to illuminate a static 2D barcode on the drug cartridge, positioned using a mechanical feature on the drug cartridge, and read the data it contains. This integrated circuit may communicate with the microcontroller 302 using a serial peripheral interface bus. Such a circuit may be activated and deactivated by the microcontroller 302 e.g., to reduce power consumption when the circuit is not needed, for example by extinguishing the cartridge illumination when data is not being read.

As previously mentioned, a sounder 330 may also be provided in the drug delivery device 10. Such a sounder may comprise a Star Micronics MZT03A. The proposed sounder may be used to provide an audible signal to the user. The sounder 330 may be driven by a pulse-width modulation (PWM) output from the microcontroller 302. In an alternative configuration, the sounder may play polyphonic tones or jingles and play stored voice commands and prompts to assist the user in operating or retrieving information from the device.

The control unit 300 further comprises a first motor driver 332 and a second motor driver 334. The motor drive circuitry may comprise Freescale MPC 17533 and is controlled by the microcontroller 302. For example, where the motor drive comprises a stepper motor drive, the drive may be controlled using general purpose digital outputs. Alternatively, where the motor drive comprises a brushless DC motor drive, the drive may be controlled using a Pulse Width Modulated (PWM) digital output. These signals control a power stage, which switches current through the motor windings. The power stage requires continuous electrical commutation. This may for example increase device safety, decreasing the probability of erroneous drug delivery.

The power stage may consist of a dual H-bridge per stepper motor, or three half-bridges per brushless DC motor. These may be implemented using either discrete semiconductor parts or monolithic integrated circuits.

The control unit 300 further comprises a first and a second motor 336, 338, respectively. As explained in greater detail below, the first motor 336 may be used to move the stopper 94 in the first cartridge 90. Similarly, the second motor 338 may be used to move the stopper 104 in the second cartridge. The motors can be stepper motors, brushless DC motors, or any other type of electric motor. The type of motor may determine the type of motor drive circuit used. The electronics for the device may be implemented with one main, rigid printed circuit board assembly, potentially with additional smaller flexible sections as required, e.g., for connection to motor windings and switches.

The microcontroller provided on the PCBA 350 will be programmed to provide a number of features and carry out a number of calculations. For example, and perhaps most importantly, the microcontroller will be programmed with an algorithm for using a certain therapeutic dose profile to calculate at least a dose of the secondary medicament based at least in part on the selected dose of the primary medicament.

For such a calculation, the controller may also analyze other variables or dosing characteristics in calculating the amount of second medicament to administer. For example, other considerations could include at least one or more of the following characteristics or factors:

a. Time since last dose;

b. Size of last dose;

c. Size of current dose;

d. Current blood glucose level;

e. Blood glucose history;

f. Maximum and/or minimum permissible dose size;

g. Time of day;

h. Patient's state of health;

i. Exercise taken; and

j. Food intake.

These parameters may also be used to calculate the size of both the first and the second dose size

In one arrangement, and as will be described in greater detail below, a plurality of different therapeutic dose profiles may be stored in the memory device or devices operatively coupled to the microcontroller. In an alternative arrangement, only a single therapeutic dose profile is stored in the memory device operatively coupled to the microcontroller.

The presently proposed electromechanical drug delivery device is of particular benefit to patients with dexterity or computational difficulties. With such a programmable device, the single input and associated stored predefined therapeutic profile removes the need for the user or patient to calculate their prescribed dose every time they use the device. In addition, the single input allows easier dose setting and dispensing of the combined compounds.

In addition to computing the dose of the second medicament, the microcontroller can be programmed to achieve a number of other device control operations. For example, the microcontroller may be programmed so as to monitor the device and shut down the various elements of the system to save electrical energy when the device is not in use. In addition, the controller can be programmed to monitor the amount of electrical energy remaining in the battery 306. In one preferred arrangement, an amount of charge remaining in the battery can be indicated on the digital display 80 and a warning may be given to the user when the amount of remaining battery charge reaches a predetermined threshold level. In addition, the device may include a mechanism for determining whether there is sufficient power available in the battery 306 to deliver the next dose, or it will automatically prevent that dose from being dispensed. For example, such a monitoring circuit may check the battery voltage under different load conditions to predict the likelihood of the dose being completed. In a preferred configuration the motor in an energized (but not moving) condition and a not energized condition may be used to determine or estimate the charge of the battery.

Preferably, the drug delivery device 10 is configured to communicate via a data link (i.e., either wirelessly or hard wired) with various computing devices, such as a desktop or laptop computer. For example, the device may comprise a Universal Serial Bus (USB) for communicating with a PC or other devices. Such a data link may provide a number of advantages. For example, such a data link may be used to allow certain dose history information to be interrogated by a user. Such a data link could also be used by a health care professional to modify certain key dose setting parameters such as maximum and minimum doses, a certain therapeutic profile, etc. The device may also comprise a wireless data link, for example an IRDA data link or a Bluetooth data link. In an alternative embodiment, a preferred Bluetooth module comprises a Cambridge Silicon Radio (CSR) Blue core 6.

In an example embodiment, the device has USB On-The-Go (USB OTG) capability. USB OTG may allow the drug delivery device 10 to generally fulfill the role of being slave to a USB host (e.g., to a desktop or notebook computer) and to become the host themselves when paired with another slave device (e.g. a BGM).

For example, standard USB uses a master/slave architecture. A USB Host acts as the protocol master, and a USB ‘Device’ acts as the slave. Only the Host can schedule the configuration and data transfers over the link. The Devices cannot initiate data transfers, they only respond to requests given by a host. Use of OTG in the drug delivery device 10 introduces the concept that the drug delivery device can switch between the master and slave roles. With USB OTG, the device 10 at one time be a ‘Host’ (acting as the link master) and a ‘Peripheral’ (acting as the link slave) at another time.

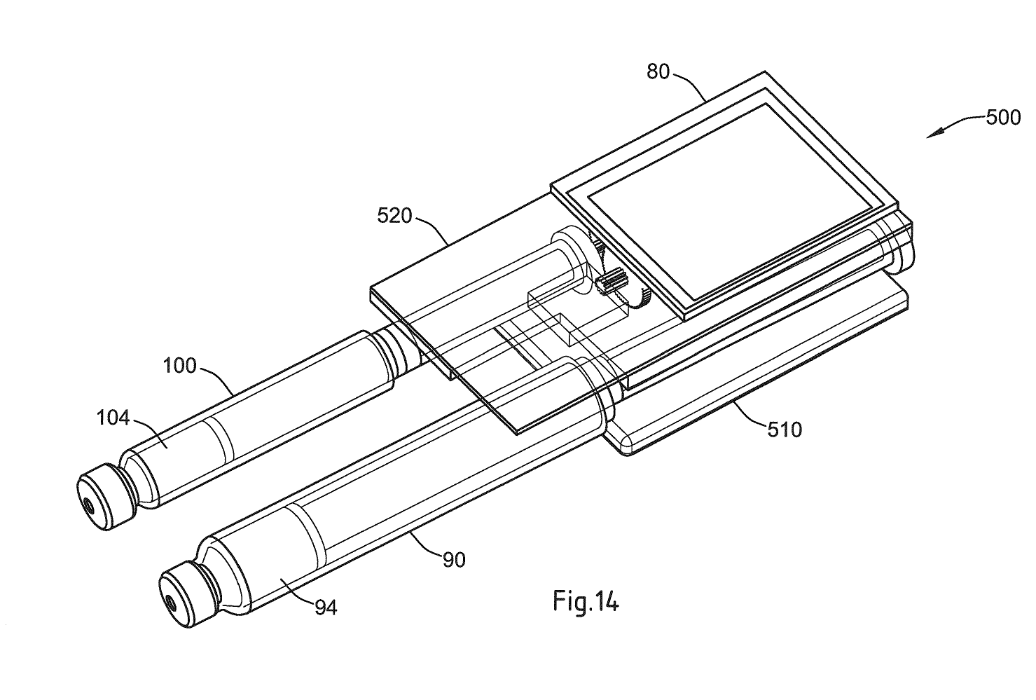

FIG. 14 illustrates various internal components of the drug delivery device 10 illustrated in FIG. 1 including one preferred arrangement of a drive train 500. As illustrated, FIG. 14 illustrates the digital display 80, a printed circuit board assembly (PCBA) 520 (such as the PCB 350 illustrated in FIG. 13), along with a power source or battery 510. The PCBA 520 may be positioned between the digital display 80 and a drive train 500 with the battery or power source 510 positioned beneath this drive train. The battery or power source 510 is electronically connected to provide power to the digital display 80, the PCBA 520 and the drive train 500. As illustrated, both the first and second cartridges 90, 100 are shown in an expended state. That is, the first and second cartridges are illustrated in an empty state having a stopper at a most distal position. For example, the first cartridge 90 (which ordinarily contains the first medicament 92) is illustrated as having its stopper 94 in the distal position. The stopper 104 of the second cartridge 100 (ordinarily containing the second medicament 102) is illustrated in a similar position.

With reference to FIG. 14, it may be seen that there is provided a first region defining a suitable location for a power source 510 such as a replaceable battery or batteries. The power source 510 may comprise a rechargeable power source and may be recharged while the power source 510 remains in the device. Alternatively, the power source 510 may be removed from the drug delivery device 10 and recharged externally, for example, by way of a remote battery charger. This power source may comprise a Lithium-Ion or Lithium-polymer power source. In this preferred arrangement, the battery 510 comprises a generally flat and rectangular shaped power source.

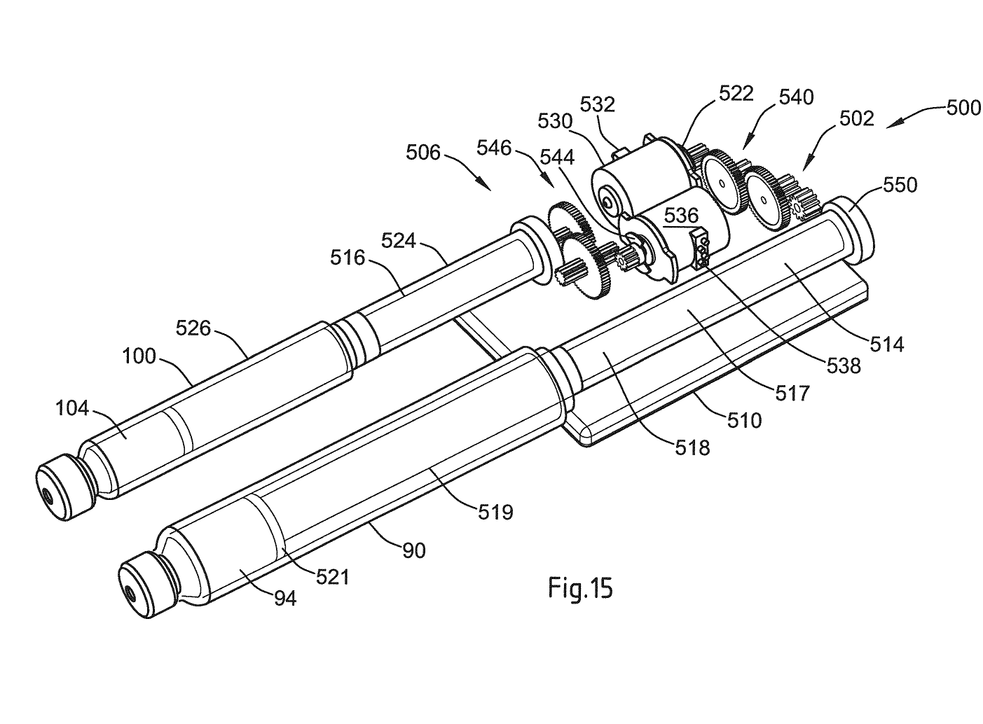

FIG. 15 illustrates the first arrangement of the electro-mechanical system illustrated in FIG. 14 with both the digital display 80 and the PCBA 520 omitted. As illustrated in FIG. 15, the electro-mechanical system 500 operates to expel a dose from the first cartridge 90 containing the primary medicament 92 and the second cartridge 100 containing the secondary medicament 102. Again, as illustrated in FIG. 15, the first and second cartridges 90, 100 are illustrated in an empty state having stoppers at a most distal position.

In this preferred electro-mechanical system 500, the system comprises an independent mechanical driver for each cartridge 90, 100. That is, an independent mechanical driver 502 operates to expel a dose from the first cartridge 90 and an independent mechanical driver 506 operates to expel a dose from the second cartridge 100. In an alternative electro-mechanical system 500 operating on three different medicaments, three independent mechanical drivers could be provided. The independent mechanical drivers act under control of the motor drivers 332, 334 of the control unit 300 (see, e.g., FIG. 12).

The first independent mechanical driver 502 operates to expel a dose from the first cartridge 90. This first driver 502 comprises a first motor 530 that is operatively coupled to a first gearing arrangement 540. To energize this motor 530, a connector 532 is provided as a means of electrically connecting to the motor driver 332. This first gearing arrangement 540 is mechanically linked to a proximal portion of the first telescoping piston rod 514. The first telescoping piston rod 514 is illustrated in a fully extended position having a distal end 521 acting on the stopper 94 of the first cartridge 90.

As this gearing arrangement 540 is driven by the output shaft of the first motor 530, this arrangement 540 rotates the proximal portion 518 of the first telescoping piston rod 514. As this proximal portion 518 of the piston rod 514 is rotated, the second or distal portion 519 of the piston rod 514 is driven in a distal direction.

Preferably, the proximal portion 518 of the telescope piston rod 514 comprises an external thread 517. This thread 517 engages the distal portion 519 which has in integrated nut comprising a short threaded section at a proximal end of the distal portion 519. This distal portion 519 is prevented from rotating via a key acting in a keyway. Such a keyway may pass through the middle of first telescope 514. Therefore, when the first gearbox arrangement 540 causes rotation of the proximal section 518, rotation of the proximal portion 518 acts upon the distal end 521 to thereby drive the distal portion of telescope piston rod to extend along the longitudinal axis.

Moving in this distal direction, the distal end 521 of the second portion 519 of the piston rod 514 exerts a force on a stopper 94 contained within the first cartridge 90. With this distal end 521 of the piston rod 514 exerting a force on the stopper, the user selected dose of the first medicament 92 is forced out of the cartridge 90 and into an attached dispense interface 200 and consequently out an attached needle assembly 400 as previously discussed above.

A similar injection operation occurs with the second independent driver 506 when the controller first determines that a dose of the second medicament 102 is called for and determines the amount of this dose. As previously mentioned, in certain circumstances, the controller may determine that a dose of the second medicament 102 may not be called for and therefore this second dose would be “set” to a “0” dose.

Preferably, motors 530, 536 comprise motors suitable for electronic commutation. Most preferably, such motors may comprise either a stepper motor or a brushless DC motor.

To inject a dose of the primary and secondary medicaments 92, 102, a user will first select a dose of the primary medicament by way of the human interface components on the display 80. (see, e.g., FIGS. 1 and 3). After a dose of the drug from the primary medicament 92 has been selected, the microcontroller will utilize a previously stored algorithm for determining the dose size of a second drug 102 from a second medicament cartridge. This pre-defined algorithm may help to determine at least in part the dose of the second medicament 102 based on a pre-selected therapeutic profile. In one arrangement, these therapeutic profiles are user selectable. Alternatively, these therapeutic profiles may be password protected and selectable only by a person authorized with the password, such a physician or patient care giver. In yet another arrangement, the therapeutic profile may only be set by the manufacture or the supplier of the drug delivery device 10. As such, the drug delivery device 10 may be provided with only one profile.

When the dose sizes of the first and second medicaments have been established, the user can press the injection button 74 (see e.g., FIG. 3). By pressing this button 74, the motor drivers 332, 334 energize both the first and the second motors 530, 536 to begin the injection process described above.

The piston rods 514, 516 are preferably movable between a first fully withdrawn position (not shown) and a second fully extended portion (as shown in FIGS. 14 and 15). With the piston rods 514, 516 in the withdrawn position, the user will be allowed to open up the respective cartridge retainer and remove an empty cartridge. In one preferred arrangement, an end stop switch may be provided in the main body 14 of the drug delivery device 10 so as to detect when either or both of the piston rods 514, 516 are in a fully withdrawn position. Tripping of the end stop switch may release a catch or other fastening device so as to allow access to the main body for replacement of either cartridge 90, 100.

In one preferred arrangement, both the first and second motors 530, 536 operate simultaneously so as to dispense the user selected dose of the first medicament 92 and the subsequently calculated dose of the second medicament 102 simultaneously. That is, both the first and the second independent mechanical drivers 502, 506 are capable of driving the respective piston rods 514, 516 either at the same or a different time. In this manner, now referring to the dispense interface 200 previously discussed, the first medicament 92 enters the holding chamber 280 of the dispense interface 200 at essentially the same time as the second medicament. One advantage of such an injecting step is that a certain degree of mixing can occur between the first and second medicament 92, 102 prior to actual dose administration.

If after an injection, the patient determines that one or more of the cartridges 90,100 is spent and therefore needs to be exchanged, the patient can follow the following method of cartridge exchange:

a. Remove the double ended needle from the dispense interface 200;

b. Remove the dispense interface 200 from the cartridge holder 40 of the device 10;

c. Enable a menu option on the digital display 80 to change the first cartridge 90 and/or the second cartridge 100;

d. Rewind the first and/or the second piston rods 514, 516;

e. The first and/or second cartridge retainer doors will pop open;

f. The user removes the spent cartridge and replaces this spent cartridge with a new cartridge;

g. The reservoir doors may manually be closed;

h. Once the doors are closed, the first and second piston rods 514, 516 advance so that a most distal portion of each rod will meet the stopper of the respective cartridge and will stop advancing when a bung detect mechanism coupled to the microcontroller is activated;

i. The user replaces the dispense interface 200 in the one way manner on the cartridge holder 40;

j. The user can, optionally, connect a new double ended needle to the dispense interface 200;

k. The user can, optionally, perform a test shot or a priming step with the device 10; and

l. The user can then set the next dose for a subsequent dose administration step.

One or more of the steps may be performed automatically, for example controlled by microcontroller 302, such as the step of rewinding the first and/or second piston rod.

In an alternative arrangement, the controller may be programmed so that the first and the second independent mechanical drivers 502, 506 may be operated to dispense either the first medicament 92 or the second medicament 102 prior to the other medicament. Thereafter, the second or the primary medicament may then be dispensed. In one preferred arrangement, the secondary medicament 102 is dispensed before the primary medicament 92.

Preferably, the first and second motors 530, 536 comprise electronic commutation. Such commutation may help to minimise the risk of a motor runaway condition. Such a motor runaway condition could occur with a system comprising a standard brushed motor experiencing a fault. In one embodiment of the motor drive system, a watchdog system may be provided. Such a system has the ability to remove power to either or both of the motors in the event of a software malfunction or a failure of the electronic hardware. To prevent the power from being removed, the correct input from a number of sections of the electronic hardware and/or the microcontroller software will need to be provided. In one of these input parameters is incorrect; power may be removed from the motor.

In addition, preferably both motors 530, 536 may be operated in a reverse direction. This feature may be required in order to allow the piston rods 514, 516 to be moved between a first and a second position.

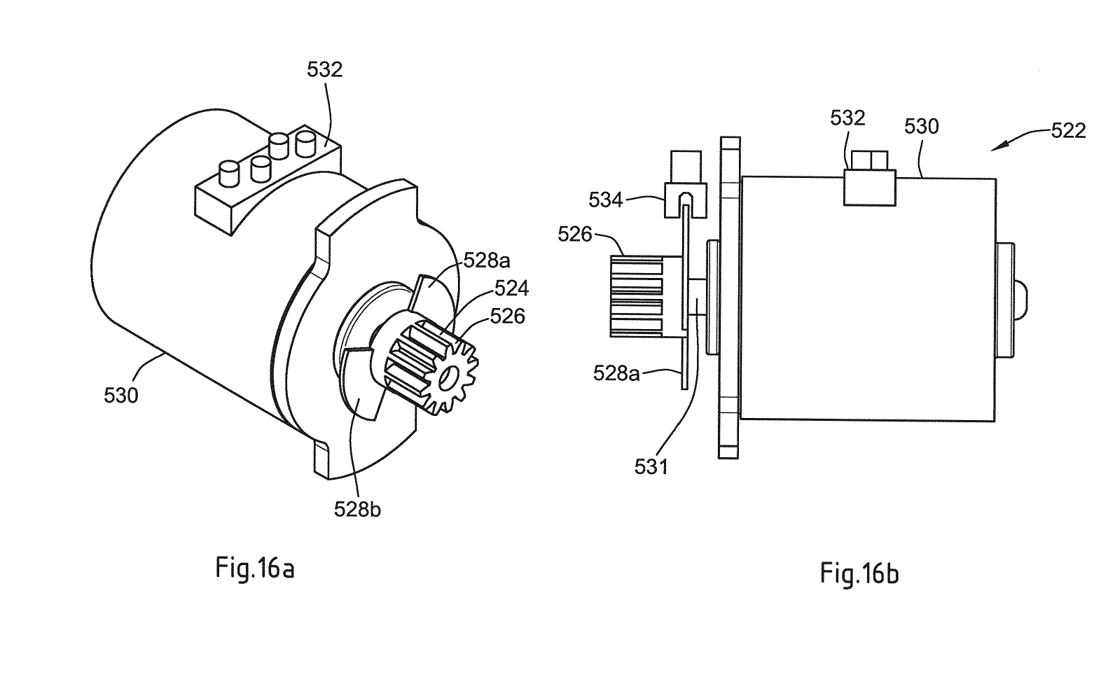

Preferably, the first independent drive train 502 illustrated in FIG. 15 comprises a first motion detection system 522. FIG. 16a illustrates a perspective view of the first motor 530 illustrated in FIG. 15. FIG. 16b illustrates a preferred motion detection system 522 comprising the first motor 530 illustrated in FIG. 16a in conjunction with a digital encoder 534.

As illustrated in FIGS. 16a and 16b, such a motion detection system 522 may be beneficial as it can be utilized to provide operational and positional feedback from the first independent driver 502 to the control unit of the drug delivery device 10. For example, with respect to the first independent driver 502, a preferred motion detection system 522 may be achieved through the use of a first motor pinion 524. This first pinion 524 operatively coupled to an output shaft 531 of the first motor 530. The first pinion 524 comprises a rotating gearing portion 526 that drives a first gear of the first gearing arrangement 540 (see, e.g., FIG. 15). The first motor pinion 524 also comprises a plurality of flags 528a-b. In this first motion detection system arrangement 522, the first pinion 524 comprises a first flag 528a and a second flag 528b. These two flags 528a-b are positioned on the motor pinion 524 so that they pass through a first optical encoder 534 as the motor output shaft 531 and hence the connected first pinion 524 rotate when the motor is driven.

Preferably, as the first and second flags 528a-b pass through the first optical encoder 534, the encoder 534 can send certain electrical pulses to the microcontroller. Preferably, the optical encoder 534 sends two electrical pulses per motor output shaft revolution to the microcontroller. As such, the microcontroller can therefore monitor motor output shaft rotation. This may be advantageous to detect position errors or events that could occur during a dose administration step such as jamming of the drive train, incorrect mounting of a dispense interface or needle assembly, or where there is a blocked needle.

Preferably, the first pinion 524 comprises a plastic injection molded pinion. Such a plastic injection molded part may be attached to the output motor shaft 531. The optical encoder 534 may be located and attached to a gearbox housing. Such a housing may contain both the first gearing arrangement 540 along with the optical encoder 534. The encoder 534 is preferably in electrical communication with the control unit potentially via a flexible portion of the PCB. In a preferred arrangement, the second independent drive train 506 illustrated in FIGS. 14 and 15 comprises a second motion detection system 544 that operates in a similar fashion as the first motion detection system 522 of the first drive train 502.

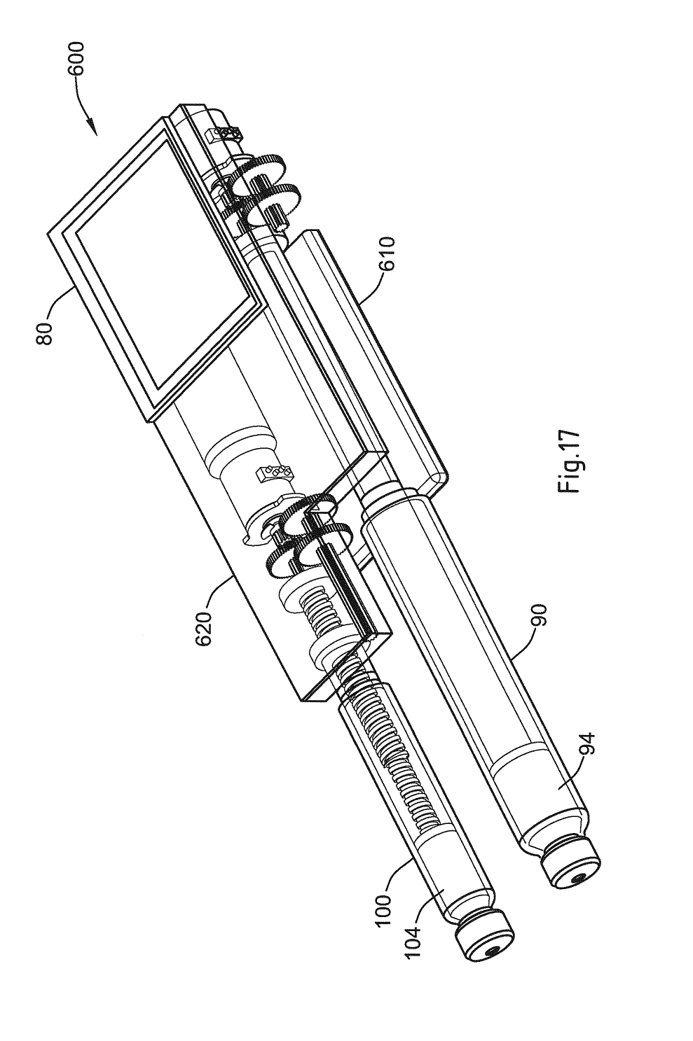

FIG. 17 illustrates various internal components of the drug delivery device 10 illustrated in FIG. 1 including a preferred alternative drive train arrangement 600. As illustrated, FIG. 17 illustrates the digital display 80, a printed circuit board assembly (PCBA) 620, along with a power source or battery 610. The PCBA 620 may be positioned between the digital display 80 and a drive train 600 with the battery or power source 610 positioned beneath this drive train. The battery or power source 610 is electronically connected to provide power to the digital display 80, the PCBA 620 and the drive train 600. The digital display 80 and the PCBA 620 of this alternative drive train arrangement 600 operate in a similar manner as previously described.

As illustrated, both the first and second cartridges 90, 100 are shown in an expended state. That is, the first and second cartridges are illustrated in an empty state having a stopper at a most distal position. For example, the first cartridge 90 (which ordinarily contains the first medicament 92) is illustrated as having its stopper 94 at the end or most distal position. The stopper 104 of the second cartridge 100 (ordinarily containing the second medicament) is illustrated in a similar end position.

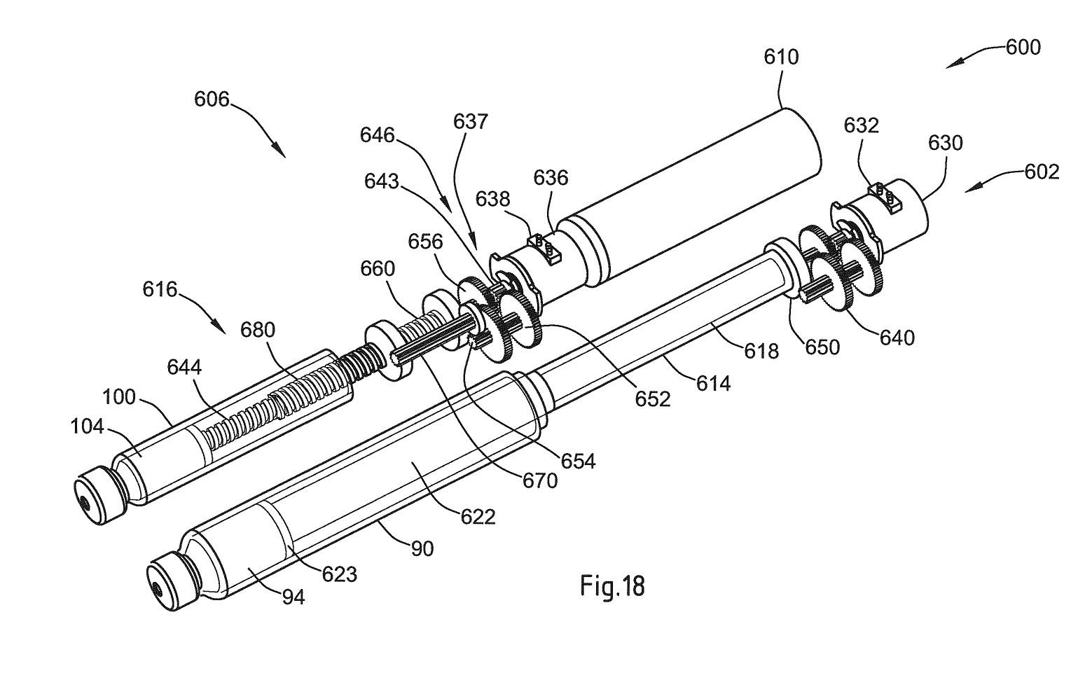

FIG. 18 illustrates the electro-mechanical system illustrated in FIG. 17 with both the digital display 80 and the PCBA 620 omitted. As illustrated, this alternative electro-mechanical system 600 operates to expel a dose from the first cartridge 90 containing a primary medicament 92 and the second cartridge 100 containing a secondary medicament 102. In this preferred electro-mechanical system 600, the system comprises an independent mechanical driver for both the first cartridge and the second cartridge. That is, an independent mechanical driver 602 operates to expel a dose from the first cartridge 90 and an independent mechanical driver 606 operates to expel a dose from the second cartridge 100. If this preferred electro-mechanical system 600 were to be reconfigured to operate on three different medicaments contained within three separate cartridges, three independent mechanical drivers could be provided so as to administer a combined dose. The independent mechanical drivers act under control of the motor drivers 332, 334 of the control unit 300 (see, e.g., FIG. 12).

The first independent mechanical driver 602 operates to expel a dose from the first cartridge 90 and operates in a similar manner as the independent drivers 502, 506 described with reference to the drive train 500 illustrated in FIGS. 14-15 above. That is, this first independent driver 602 comprises a first motor 630 that is operatively coupled to a first gearing arrangement 640. To energize this motor 630, a connector 632 is provided as a means of electrically connecting to the motor driver 332. This first gearing arrangement 640 is mechanically linked to a proximal portion of the telescoping piston rod 614. As this gearing arrangement 640 is driven by an output shaft of the first motor 632, this arrangement 640 rotates the proximal portion 618 of the telescoping piston rod 614. As this proximal portion 618 of the piston rod 614 is rotated, the second or distal portion 622 of the piston rod 614 is driven in a distal direction. Moving in this distal direction, a distal end 623 of the second portion 622 of the piston rod 614 exerts a force on the stopper 94 contained within the first cartridge 90. With a distal end 623 of the piston rod 614 exerting a force on the stopper 94, the user selected dose amount of the first medicament 92 is forced out of the cartridge 90 and into an attached dispense interface 200 and consequently out an attached needle assembly 400 as previously discussed.

Preferably, the first independent mechanical driver 602 comprises a bung or stopper detection system. Such a detection system may be used detect the position of the cartridge stopper 94 following a cartridge change event. For example, when a cartridge change event occurs, the piston rod is retracted in a proximal position so as to enable a user to open the cartridge retainer and thereby provide access to a spent cartridge. When the cartridge is replaced and the cartridge retainer door is shut, the piston rod will advance in a distal direction towards the stopper of new the cartridge.

In one preferred stopper detection system, a switch is provided at the distal end of the piston rod. Such a switch may comprise a mechanical, optical, capacitive, or inductive type switch. Such a switch would be in communication with the microcontroller and indicates when the piston rod is in contact with the stopper and hence may be used as a mechanism for stopping the drive system.

The second independent mechanical driver 606 operates to expel a dose from the second cartridge 100 in a different manner than the first independent driver 602. That is, this second mechanical driver 606 comprises a second motor 636 that is operatively coupled to a second gearing arrangement 646. To energize this motor 636, a connector 638 is provided as a means of electrically connecting to the motor driver 334.

This independent mechanical driver 606 comprises:

a. A motor 636;

b. A second gearing arrangement 646; and

c. A telescope piston rod 616.

The second gearing arrangement 646 is mechanically linked to a proximal portion of a nested piston rod 660. As this gearing arrangement 646 is driven by the output shaft of the second motor 636, this arrangement 646 rotates the proximal portion 660 of the telescoping piston rod 616.

The second gearing arrangement 646 comprises a motor pinion along with a plurality of compound gears (here four compound gears) along with a telescope input piston rod. Two of the compound gears are elongated to enable continuous mesh engagement with the input piston rod as the telescope extends in a distal direction to exert an axially pressure on the cartridge stopper 104 so as to expel a dose from the cartridge. The elongated gear may be referred to as a transfer shaft. The gearbox arrangement preferably has a ratio of 124:1. That is, for every revolution of the telescope input screw the output shaft of the second motor rotates 124 times. In the illustrated second gearing arrangement 646, this gearing arrangement 646 is created by way of five stages. As those skill in the art will recognize, alternative gearing arrangements may also be used.

The second gearing arrangement 646 comprises three compound reduction gears 652, 654, and 656. These three compound reduction gears may be mounted on two parallel stainless steel pins. The remaining stages may be mounted on molded plastic bearing features. A motor pinion 643 is provided on an output shaft of the second motor 636 and is retained on this shaft 637, preferably by way of an interference or friction fit connection.

As described above, the motor pinion 643 may be provided with two mounted “flag” features that interrupt the motion detect optical sensor. The flags are symmetrically spaced around the cylindrical axis of the pinion.

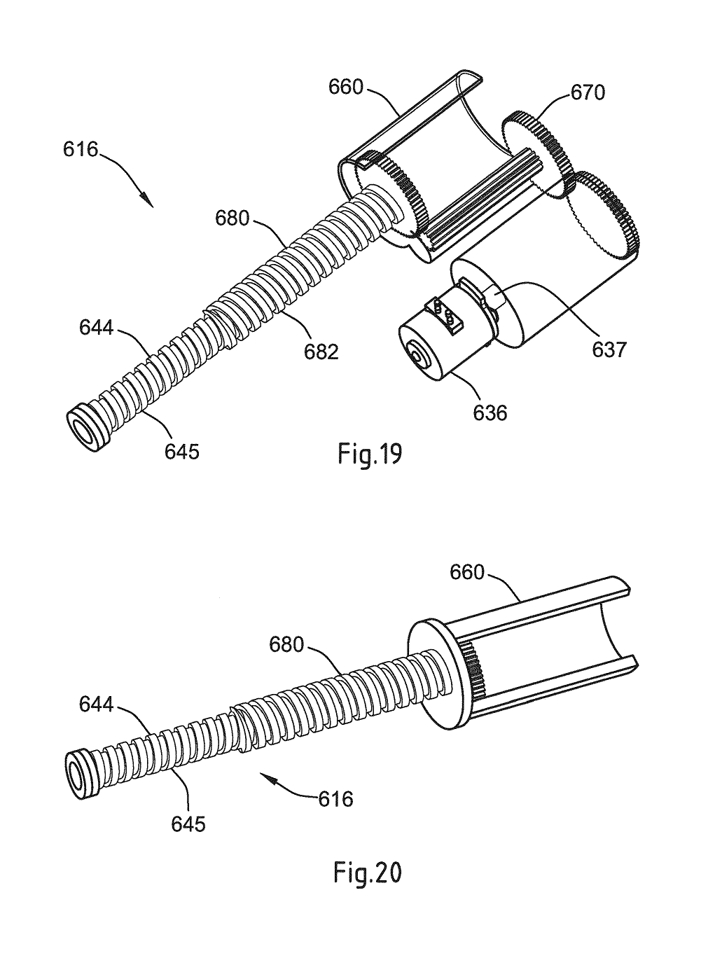

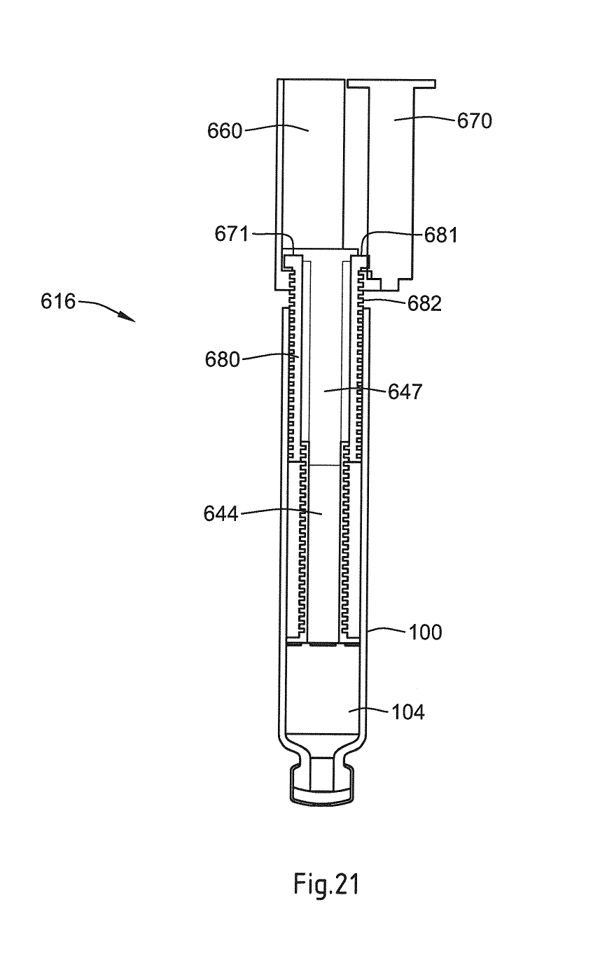

The drive train telescoping piston rod 616 is illustrated in FIG. 19 and comprises a telescope plunger 644 that is operatively coupled to an input screw 680. FIG. 20 illustrates a perspective view of the telescope piston rod 616 coupled to a latch barrel. FIG. 21 illustrates a cross sectional view of the independent mechanical driver with the piston rod 616 in an extended position.

As illustrated, the outer elements (the telescope piston rod plunger 644 and telescope) create the telescopic piston rod 616 and react to the compressive axial forces that are developed. An inner element (telescope piston rod key 647 provides a means of reacting the rotational input force. This operates with a continuous motion and force since there will be no changes in drive sleeve diameter to generate varying levels of force.

The transfer shaft 670 is operatively linked to the gearing arrangement 646. The transfer shaft 670 can rotate but it cannot move in an axial direction. The transfer shaft 670 interfaces with the second gearing arrangement 646 and transfers the torque generated by the second gearbox arrangement 646 to the telescope piston rod 616.

Specifically, when the transfer shaft 670 is rotated by way of the gearing arrangement 646, the transfer shaft 670 will act on an integrated geared part 681 on a proximal end of the input screw 680. As such, rotation of the transfer shaft 670 causes the input screw 680 to rotate about its axis.

A proximal portion of the input screw 680 comprise a threaded section 682 and this threaded section is mated with a threaded section of the latch barrel 660. As such, when the input screw 680 rotates, it winds or screws itself in and out of the latch barrel 660. Consequently, as the input screw 680 moves in and out of the latch barrel, the screw 680 is allowed to slide along the transfer shaft 670 so that the transfer shaft and the gears remain mated.

The telescope plunger 644 is provided with a threaded section 645. This threaded section 645 is threaded into short section in distal end of the input screw 680. As the plunger 644 is constrained from rotating, it will wind itself in and out along the input screw 680.

A key 647 is provided to prevent the plunger 644 from rotating. This key 647 may be provided internal to the input screw 680 of the piston rod 616. During an injection step, this key 647 moves in the axial direction towards the stopper 104 of the cartridge 100 but does not rotate. The key 647 is provided with a proximal radial peg that runs in a longitudinal slot in the latch barrel 660. Therefore, the key 647 is not able to rotate. The key may also be provided with a distal radial peg that engage a slot in the plunger 644.

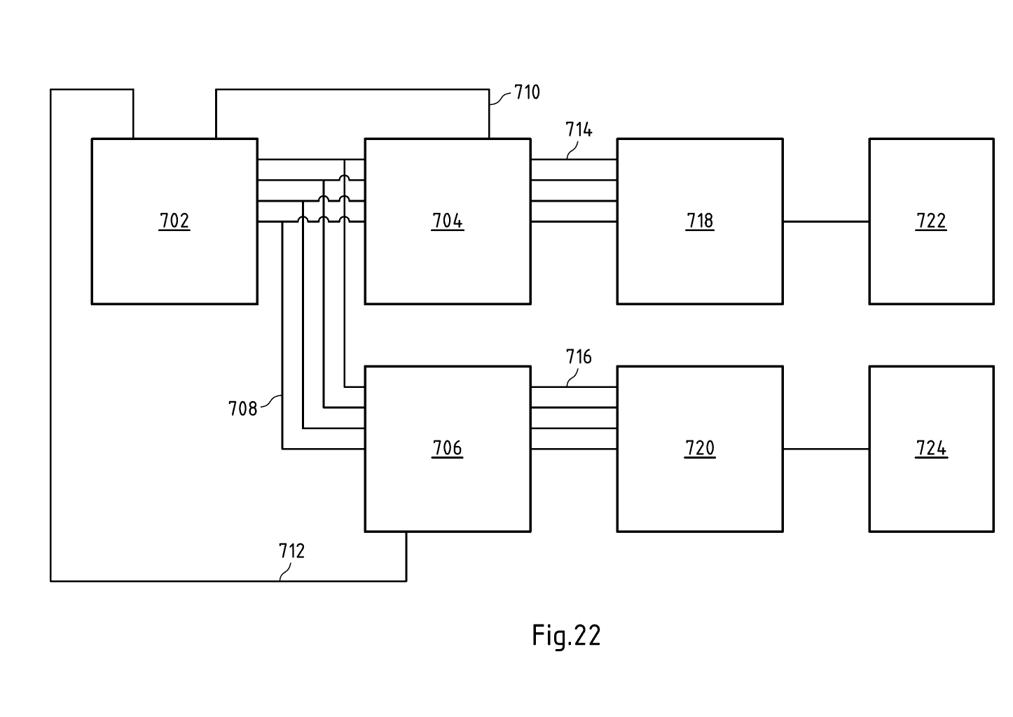

Referring now to FIG. 22, an exemplary arrangement of controlling the drive mechanism is described. Unless noted otherwise, any component referred to is identical to any one of the embodiments of that component described above with regard to FIGS. 12 to 20.

A microcontroller or other logic controller 702 is connected to a first motor driver 704 and a second motor driver 706 via a common control signal bus 708. The microcontroller 702 may be identical to the microcontroller 302 described above with reference to FIGS. 12 and 13. The first motor driver 704 and the second motor driver 706 are integrated circuits or sub-circuits composed of several components configured to receive logic signals. The first motor driver 704 may be identical to the first motor driver 332 as described above with reference to FIG. 12 and FIG. 13. The second motor driver 706 may be identical to the second motor driver 334 as described above with reference to FIG. 12 and FIG. 13.

The common control signal bus 708 is shared by the first motor driver 704 and the second motor driver 706 in the sense that any signal transmitted by the microcontroller 702 via the common control signal bus 708 is received both by the first motor driver 704 and the second motor driver 706. The common control signal bus may be a digital command transmission bus for inter-circuit communication, i.e. configured for communicating information between integrated circuits. For example, the common control signal bus may be an inter-integrated circuit (12C) bus. Thereby the microcontroller 702 may be able to program the first and second motor drivers 704, 706.

The first motor driver 704 controls a first motor 718 via a first set of motor control lines 714. The second motor driver 706 controls a second motor 720 via a second set of motor control lines 716. The first motor 718 may be identical to the first motor 336 described with reference to FIG. 3 and the second motor 720 identical to the second motor 338 described with reference to FIG. 3. Alternatively, the first motor may be identical to the first motor 530 described with reference to FIG. 16a and FIG. 16b and the second motor identical to the second motor 536.

The first motor 718 and the second motor 720 may be an electrical motor with a pinion for rotation each. The first motor 718 and the second motor 720 are configured to rotate their respective pinion in either direction.

Via the first and second set of motor control lines 714, 716, the first and second motor drivers 704, 706 control one or more current or voltage parameter such as a current or voltage value, a current or voltage peak value, or a current or voltage frequency and/or waveform through the motor coils of the first and second motors 718, 720, respectively, and set various other parameters such as the direction of rotation.

Each set of motor control lines 714, 716 may be a set of electrical lines where an operational parameter of the respective motor 718, 720 is controlled by a dedicated signal on a particular line of the respective set of motor control lines 714, 716. For example, an analogue or digital value on a first line of the first set of motor control lines 714 may determine the voltage peak value applied to the motor coils of the first motor 718. A further analogue or digital value on a second line of the first set of motor control lines may determine the frequency of the voltage applies to the motor coils of the first motor 718. The second set of motor control lines 716 works in the same way for the second motor 720. Each parameter may also be transmitted as a pulse-width modulated (PWM) signal on a dedicated line of the set of motor control lines 714, 716.

The first and second motor drivers 704, 706 are configured to do this in accordance with the signals received from the microcontroller 701 via the common control signal bus 708. In other words, the first and second motor drivers 704, 706 generate signals corresponding to the desired parameters on the set of motor control lines 714, 716 based on the higher-level commands received by the first and second motor driver 704, 706 via the common control signal bus 708. The command received via the common control signal bus 708 may comprise the command to advance the first motor 718 for two hundred rotations, for example. The first motor driver 704 would then through its internal logic determine the proper operational parameters to send to the first motor 718, their timing as well as their sequence and send the according signals via the first set of motor control lines 714. This would work the same way for the second motor 720 and the second motor driver 706.

In particular, both the first motor driver 704 and the second motor driver 706 receive the same direction control signal from the common control signal bus 708, thereby setting an identical direction of rotation of the first motor 718 and the second motor 720 via the first and second sets of motor control lines 714, 716.

The microcontroller 702 can further send a first enable signal to the first motor driver 704 via a first enable signal line 710 and also, independently, send a second enable signal to the second motor driver 706 via a second enable signal line 712. The first motor driver 704 only switches current through the motor coil of the first motor 718 when it has received a first enable signal via the first enable signal line 710. Likewise, the second motor driver 706 only switches current through the motor coil of the second motor 720 when it has received a second enable signal via the second enable signal line 712. Thereby, any of the first and second motors 718, 720 can be selectively disabled by the microcontroller 701.

In an alternative embodiment, there may only be a single enable line connected to both the first motor driver 704 and the second motor driver 706 which, for example by way of a binary enable signal, sends only a signal enable signal determining which if the first and second motor drivers 704, 706 is enabled.

The first and second motors 718, 720 are arranged facing in opposite directions. The rotation of the pinion of the first motor 718 is transmitted via a first gearing arrangement to a first drive train 722. The rotation of the pinion of the second motor 720—facing in the opposite direction of the pinion of the first motor 718—is transmitted via a second gearing arrangement to a second drive train 714. The first and second drive trains 722, 724 are arranged in parallel and facing the same direction. Each of the drive trains 722, 724 is connected via a further respective gearing arrangement to a respective telescope piston rod with which a respective stopper can be moved to expel fluid from a respective cartridge. The first drive train 722 may be connected via the respective gearing arrangement to the first telescope piston rod 614 and the second drive train may be connected via the respective gearing arrangement to the second telescope piston rod 616, both described with reference to FIG. 18.

If only one of the motor drivers 704, 706 is enabled via the enable signal lines 710, 712, only the motor of the enabled motor driver will rotate. The other motor will not rotate.

However, if both motor drivers 704, 706 are enabled via the enable signal lines 710, 712, both motors 718, 720 can only rotate in the same direction, i.e. either both clockwise or both counter-clockwise, because the first and second motor drivers 704, 706 receive the same direction control signal.

Since the first motor 718 is oriented in a direction opposite to that of the second motor 720, it follows that when both motors 718, 720 rotate, the first drive train 722 and the second drive train 724 must rotate in opposite directions.

Since further one direction of rotation of the drive trains 722, 724 corresponds to an advancing of their associated stopper, i.e. an expulsion of fluid from the cartridge, and the other direction corresponds to a retraction of the associated piston rod a simultaneous expulsion of fluid from both cartridges by simultaneously advancing stoppers is effectively prevented.

This holds true even if the signals on the common control signal bus 708, the first enable signal line 710 and the second enable signal line 712 are utterly undefined.

In an alternative embodiment, the first motor 718 is arranged in the same direction as the second motor 720. However, the first gearing arrangement may cause an inversion of the rotational movement, for example by having an additional gear or a screw thread with opposite sense of rotation. Thus, the first gearing arrangement is configured to provide a rotation of the first drive train causing an advancing of the first piston rod, while the second gearing arrangement is configured to provide a rotation of the second drive train causing a retraction of the second piston rod. Thus, when the first and the second motors receive the same control signals and rotate in the same direction, one piston rod will advance while the other will retract.

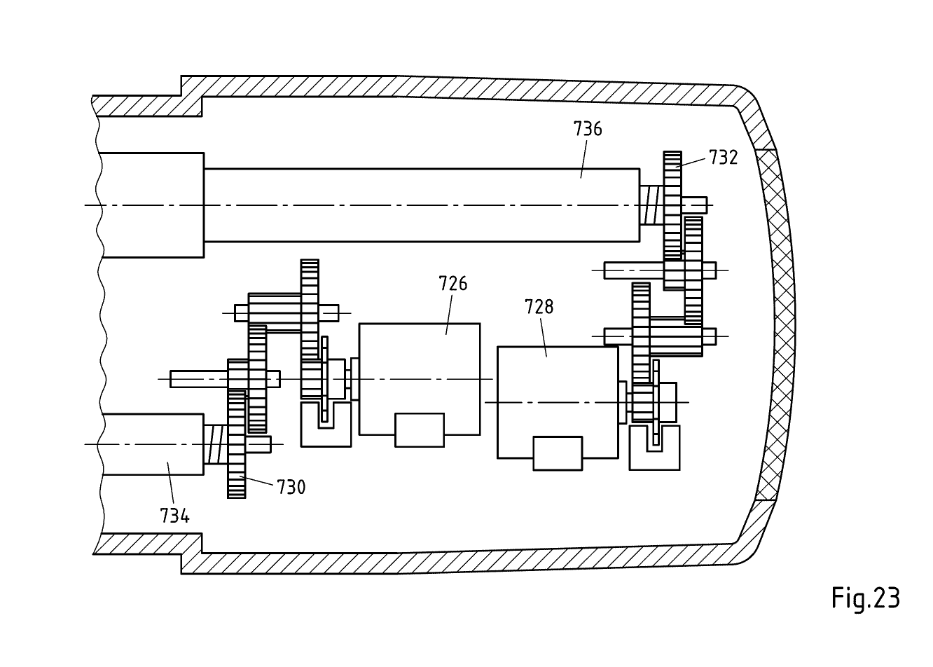

Referring now to FIG. 23, a schematic view of an exemplary motor component corresponding to the block diagram as illustrated in FIG. 22 and described with reference thereto is presented. The first driving motor 726 is arranged opposite to the second driving motor 728. The rotation of the pinion of the first driving motor 726 is transmitted via a first gearing arrangement 730 to a first drive train 734.

Likewise, the rotation of the pinion of the second driving motor 728 is transmitted via a second gearing arrangement 732 to a second drive train 736. When the first driving motor 726 and the second driving motor 728 are rotating in the same direction, the first drive train 734 and the second drive train 736 are rotating in opposite directions.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the aforementioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

1-15. (canceled)

16. An apparatus comprising:
a first drive shaft,
a second drive shaft, and
a motor component configured to receive a control signal, which control signal comprises a direction control signal,
the motor component further configured to selectively drive the first drive shaft and the second drive shaft based on the received control signal, and
further configured to drive the first drive shaft and the second drive shaft in a respective direction based on the direction control signal, such that a direction control signal for driving the first drive shaft in a first direction is a direction control signal for driving the second drive shaft in a direction opposite to the first direction.

17. The apparatus of claim 16, wherein the first drive shaft and the second drive shaft are arranged in parallel.

18. The apparatus of claim 16, further comprising:
a first mechanism for linearly moving a stopper,
a second mechanism for linearly moving a stopper,
a first gearing arrangement configured to couple the first drive shaft to the first mechanism for linearly moving a stopper, and
a second gearing arrangement configured to couple the second drive shaft to the second mechanism for linearly moving a stopper,
wherein the first and second mechanisms for linearly moving a stopper and the first and second gearing arrangements are configured such that the first and second mechanism for linearly moving a stopper, respectively, engages in an extending action when the first or second drive shaft, respectively, is driven in a first direction and engages in a retracting action when the first or second drive shaft, respectively, is driven in a direction opposite to the first direction.

19. The apparatus of claim 18, wherein the first mechanism for linearly moving a stopper is configured to expel liquid from a first cartridge by engaging in the extending action and the second mechanism for linearly moving a stopper is configured to expel liquid from a second cartridge by engaging in the extending action.

20. The apparatus of claim 19, wherein the first mechanism for linearly moving a stopper and the second mechanism for linearly moving a stopper are arranged in parallel.

21. The apparatus of claim 18, wherein the first and second mechanisms for linearly moving a stopper comprise a first telescope piston rod and a second telescope piston rod, respectively.

22. The apparatus of claim 16, wherein the motor component comprises:
a first motor configured to drive the first drive shaft, and
a second motor configured to drive the second drive shaft.

23. The apparatus of claim 22, wherein the first and second motors are each configured to selectively rotate in a clockwise direction or in a counter-clockwise direction.

24. The apparatus of claim 23, wherein the first motor is arranged anti-parallel to the second motor such that a rotation of the first motor in the same direction as the rotation of the second motor causes the first drive shaft and the second drive shaft to be driven in opposite directions.

25. The apparatus of claim 24, further comprising a casing comprising
a proximal end of the first drive shaft,
a proximal end of the second drive shaft,
the first motor, and
the second motor,
wherein the first drive shaft is shorter than the second drive shaft, wherein the first motor and the second motor are arranged at the proximal end of the first drive shaft, at an offset towards the axis of the second drive shaft and arranged in parallel to the second drive shaft.

26. The apparatus of claim 16, comprising a control unit configured to generate the control signal and the direction control signal.

27. The apparatus of claim 26, wherein the motor component comprises:
a first motor driver configured to drive the first motor based on the received control signal and the direction control signal,
a second motor driver configured to drive the second motor based on the received control signal and the direction control signal, and
at least one signal line configured to carry the control signal and the direction control signal from the control unit,
wherein the first motor driver and the second motor driver are configured to at least partially share the at least one signal line such that they receive the identical direction control signal.

28. The apparatus of claim 27, wherein the first motor driver and the second motor driver are configured such that each receives the identical control signal and direction control signal from the shared at least one signal line.

29. The apparatus of claim 27, wherein the control unit is configured to generate a first enable signal for selectively enabling the first motor driver and is configured to generate a second enable signal for selectively enabling the second motor driver.

30. Drug delivery device comprising an apparatus according to claim 16.
